(19) United States
(12) Patent Application Publication
(10) Pub. No.: US 2022/0267417 A1
(43) Pub. Date: Aug. 25, 2022

(54) ANTIBODY AGAINST THE OPRF PROTEIN OF PSEUDOMONAS AERUGINOSA, USE THEREOF AS A MEDICAMENT AND PHARMACEUTICAL COMPOSITION CONTAINING SAME

(71) Applicants: UNIVERSITÉ GRENOBLE ALPES, SAINT MARTIN D'HERES (FR); INSTITUT POLYTECHNIQUE DE GRENOBLE, GRENOBLE (FR); CENTRE NATIONAL DE LA RECHERCHE SCIENTIFIQUE, PARIS (FR)

(72) Inventors: Jean-Luc LENORMAND, MEYLAN (FR); Landry GAYET, VILLEURBANNE (FR); Géraldine MAYEUX, SAINT-ISMIER (FR)

(21) Appl. No.: 17/629,161

(22) PCT Filed: Jul. 22, 2020
(86) PCT No.: PCT/EP2020/070725

§ 371 Date: Jan. 21, 2022

(30) Foreign Application Priority Data
Jul. 23, 2019 (FR) FR1908344

(51) Int. Cl. C07K 16/12; A61P 31/04; G01N 33/569

(52) U.S. Cl. CPC C07K 16/1214; A61P 31/04; G01N 33/56911; C07K 2317/565; C07K 2317/622; C07K 2317/24; C07K 2317/14; G01N 2333/21; C07K 2317/92

(57) ABSTRACT

The invention relates to a monoclonal antibody against the OprF protein of Pseudomonas aeruginosa or to a functional fragment of this antibody. This antibody or antibody fragment is particularly useful for the preventive or curative treatment of infections with Pseudomonas aeruginosa.

### BACKGROUND OF THE INVENTION

### Field of the Invention

The present invention relates to the field of the treatment of bacterial infections, particularly infections such as those caused by bacteria of the species Pseudomonas aeruginosa.

More particularly, the present invention relates to a monoclonal antibody against the OprF protein of Pseudomonas aeruginosa or a functional fragment of this antibody. The invention also relates to a nucleic acid molecule coding for this antibody or antibody fragment, an expression vector comprising such a nucleic acid molecule and a host cell comprising such a nucleic acid molecule or such an expression vector. The invention also relates to a method for preparing an antibody or antibody fragment according to the invention, as well as to the use of this antibody or antibody fragment as a medicament, particularly for the preventive or curative treatment of Pseudomonas aeruginosa infections. The invention furthermore relates to a pharmaceutical composition containing such an antibody or antibody fragment. The invention also relates to the use of an antibody or antibody fragment according to the invention for detecting the Pseudomonas aeruginosa bacterium in a body fluid obtained from an individual, and to a kit for such a detection, containing such an antibody or antibody fragment.

### Description of the Related Art

The prevention and treatment of hospital-acquired infections represent a major concern in the hospital sector. The incidence of these infections is steadily increasing, particularly because the pathogens responsible for these infections are increasingly resistant to antibiotics.

The Pseudomonas aeruginosa bacterium represents in particular one of the major causes of hospital-acquired infections, as well as pneumonia, in hospital settings. It is estimated that Pseudomonas aeruginosa is responsible for 10% of hospital-acquired illnesses. The number of people affected by this pathogen is very large, and the associated mortality rate is high in people with impaired immune defenses. Pseudomonas aeruginosa is an opportunistic bacterium, which is particularly involved in acute and chronic infections in patients under artificial ventilation and those with cystic fibrosis, and which is responsible for septicemia in immunocompromised patients, among which transplant patients and severely burned patients.

The Pseudomonas aeruginosa strains responsible for hospital-acquired infections are characterized by an intrinsic resistance to a wide range of antibiotics and to conventional antibiotic treatments. The bacterium is difficult to eliminate due to this antibiotic resistance and its ability to form a biofilm in patients' lungs.

However, despite the wide circulation of this pathogen and the growing number of antibiotic-resistant strains, the pharmaceutical industry has not made any effective treatment against Pseudomonas aeruginosa infections available to date.

Different therapeutic approaches have been envisaged by the prior art to develop such a treatment.

A certain number of immunogenic proteins have been identified among Pseudomonas aeruginosa, thanks to recent studies on bacterial virulence mechanisms (Chevalier et al., 2017, FEMS, 41: 698-722). These studies have revealed that these immunogens are primarily located in certain structural compartments such as flagella, pili, lipopolysaccharides, outer membrane proteins, or form part of secreted products such as mucoid exopolysaccharides, exotoxin A and proteases. Among the outer membrane proteins, the porins OprF and Oprl have been the subject of substantial research (Chevalier et al., 2017, FEMS, 41: 698-722). Hybrid proteins containing known epitopes of these proteins have been produced by fusion and tested in animal models (Weimer et al., 2009, Infect. Immun., 77(6): 2356-2366).

Another therapeutic approach envisaged by the prior art is based on the use of antibodies targeted against molecular targets conserved in all Pseudomonas aeruginosa strains. Three antibodies are thus currently under study: an anti-PcrV which targets the type III secretion system (Shionogi et al., 2016, Hum Vaccin Immunother. 12(11): 2833-2846), an anti-LPS antibody making it possible to kill the bacterium and recruit innate immune system effectors, and a bispecific antibody targeting the PcrV and PsI approaches.

However, while several therapeutic approaches are currently under development and testing, novel solutions for treating Pseudomonas aeruginosa infections must still emerge in order to address the important public health problem that Pseudomonas aeruginosa infections represent.

The aim of the present invention is thus to provide a therapeutic agent making it possible to combat bacterial infections effectively, in particular Pseudomonas aeruginosa infections, which remedies the problems associated with Pseudomonas aeruginosa antibiotic-resistance and with the lack of effective treatments against this infectious agent responsible for hospital-acquired infections and the main cause of mortality of patients with cystic fibrosis.

For this purpose, the present inventors sought to develop novel active agents of the anti-infectious antibody type, and for this they focused more specifically, as a molecular target, on the membrane protein OprF, also known as porin OprF. OprF protein is a highly abundant 38 kDa protein with a large-diameter conducting pore, involved in a large number of varied functions and which is highly conserved in all Pseudomonas aeruginosa strains (Genbank accession No.: AFM37279.1). Its important role in Pseudomonas aeruginosa virulence, described by the prior art, makes it a potential target for anti-infectious treatments.

Antibodies targeted against the OprF protein of Pseudomonas aeruginosa have been proposed by the prior art, illustrated in particular by the documents WO 2016/033547, Moon et al., Investigative Ophthalmology & Visual Science, 1988, 29: 1277-1284, and Rawling et al., 1995, Infection and Immunity, 63: 38-42. These antibodies are however obtained from inoculation of either soluble OprF protein fragments, or whole OprF protein solubilized using a detergent, i.e., in a form not corresponding to the native conformation of the protein as adopted in the bacterial membrane. These antibodies therefore cannot recognize the conformational epitopes naturally exposed by the protein in the bacterium.

### SUMMARY OF THE INVENTION

The present inventors discovered that specific antibodies, or parts of these antibodies, targeted against the OprF membrane antigen, have a particularly high biological neutralization activity in respect of the OprF protein, for all of the native linear and conformational epitopes thereof, and hence have a great therapeutic efficacy against bacterial infections due to Pseudomonas aeruginosa.

Seeking to produce antibodies binding specifically with the OprF protein of Pseudomonas aeruginosa, the present inventors developed an innovative method, which led to the discovery of antibodies having a particularly strong affinity for the protein in the native membrane form thereof.

This method consists, schematically, of producing antibodies from immune banks produced in primates from proteoliposomes containing the OprF protein of Pseudomonas aeruginosa. It is also applicable to other antigens than the porin OprF of Pseudomonas aeruginosa.

More specifically, the method for preparing antibodies, or functional antibody fragments, according to the invention comprises a step of producing proteoliposomes wherein the OprF protein of Pseudomonas aeruginosa is in its native and active form, exposing the conformational epitopes. This step can be carried out by contacting an expression vector containing the coding sequence of the OprF protein of Pseudomonas aeruginosa and a synthetic liposome, in the presence of an acellular protein synthesis system to form a reaction medium, this system enabling the transcription and simultaneous translation of the protein. The protein is then inserted into the lipid bilayer of the synthetic liposome to form a proteoliposome. Such a step is in particular described in the publication by Maccarini et al., Langmuir 2017, 33, 9988-9996. The lipid bilayer of the liposome imitating the Pseudomonas aeruginosa membrane, the OprF protein is found therein in an orientation that is suitable to have a native conformation and an open-channel conformation. More specifically, in the proteoliposomes, the OprF protein is advantageously found in its two forms, open and closed, which are naturally present in the bacterial membrane or in the vesicles released by the bacterium to counteract the immune response. In particular, the proteoliposome analyses carried out by the inventors demonstrated that the OprF protein: is found therein in the correct orientation in the liposomal membrane; is present therein in both its membrane topologies, open and closed, characterized by 8 and 16 transmembrane passages respectively; forms therein pores/channels in the liposomal membrane; is found in oligomerized form therein.

The method developed by the inventors then comprises a step of immunizing a non-human mammal, preferably a macaque (Macaca fascicularis), with these proteoliposomes, then producing a bank of antibodies, and in particular a bank of scFv fragments, from bone marrow samples from the immunized subject.

Screening this scFv bank using an expression technique, particularly using phage display, enabled the inventors to identify more than 11 sequences associated with positive clones, the complementarity determining regions of which were determined. The present inventors thus discovered that antibodies targeted against the OprF protein of Pseudomonas aeruginosa having specific sequences for the three complementarity determining regions of each of the heavy chain and light chain variable regions have particularly substantial affinity and specificity for the native epitopes of this protein, making them active substances of choice for the therapeutic treatment of Pseudomonas aeruginosa infections, especially as the primatized nature thereof makes them particularly well tolerated in humans. In the present description, the term treatment refers to both curative treatment and preventive treatment. The phenomena underpinning the obtaining of such an advantageous result will not be prejudged here. However, it may be thought, as set out above, to stem at least partially from the combination of the OprF protein expression in the proteoliposomes technique and the use of these proteoliposomes to perform immunization in the macaque. Nothing in the prior art suggested that such a combination could result in the identification of antibody regions responsible for a specific binding with a very strong affinity of these antibodies with membrane porin OprF of Pseudomonas aeruginosa, and more specifically with the native linear and conformational epitopes thereof.

Thus, according to an aspect of the present invention, a monoclonal antibody targeted against, i.e., binding specifically with, the OprF protein of Pseudomonas aeruginosa or a functional fragment of this antibody is proposed.

This antibody or functional fragment comprises:
a heavy chain variable region having the three complementarity determining regions (CDR) having the following amino acid sequences, or sequences having at least 80%, preferably at least 85%, preferably at least 90%, preferably at least 95%, preferably at least 96%, preferably at least 97%, preferably at least 98%, and preferentially at least 99%, identity with these sequences:

VH-CDR1: GYXaa₁FXaa₂Xaa₃Xaa₄G (SEQ ID NO: 1) wherein Xaa₁ is a threonine residue or a serine residue, Xaa₂ is a serine residue or an asparagine residue, Xaa₃ is an arginine residue, a serine residue or a threonine residue and Xaa₄ is a phenylalanine residue or a tyrosine residue,

VH-CDR2: INAXaa₅TGKXaa₆ (SEQ ID NO: 2) wherein Xaa₅ is a glutamic acid residue or an aspartic acid residue and Xaa₆ is an alanine residue or a serine residue,

VH-CDR3: VR,
and a light chain variable region having the three CDRs having the following amino acid sequences, or sequences having at least 80%, preferably at least 85%, preferably at least 90%, preferably at least 95%, preferably at least 96%, preferably at least 97%, preferably at least 98%, and preferentially at least 99%, identity with these sequences:

VL-CDR1: SSVXaa₇TXaa₈Xaa₉ (SEQ ID NO: 3) wherein Xaa₇ is a threonine residue, an asparagine residue, a serine residue, an alanine residue or an arginine residue, Xaa₈ is an asparagine residue, a glycine residue or a serine residue and Xaa₉ is a tyrosine residue or a phenylalanine residue,

VL-CDR2: Xaa₁₀TS wherein Xaa₁₀ is a glycine residue, an arginine residue or an alanine residue,

VL-CDR3: QQGXaa₁₁Xaa₁₂Xaa₁₃ (SEQ ID NO: 4) wherein Xaa₁₁ is a histidine residue or an asparagine residue, Xaa₁₂ is a serine residue or a threonine residue and Xaa₁₃ is a valine residue or an isoleucine residue.

An antibody, in the sense of the present invention, denotes conventionally a glycoprotein composed of two types of glycopolypeptide chains, known as heavy chain and light chain, an antibody consisting of two heavy chains and two light chains, bound by disulfide bridges. Each chain consists of a variable region and a constant region. The heavy chain variable region VH and light chain variable region VL each have three hypervariable zones, known as complementarity determining regions (CDR). Thus, in the present description, “complementarity determining region” conventionally denotes each of the three hypervariable regions of the heavy and light chain variable regions of an antibody, which form the elements of the paratrope and make it possible to determine the complementarity of the antibody with the antigen epitope. These three hypervariable regions are framed by four constant regions which form the framework (FR regions) and give the variable domain a stable configuration. The CDRs of an antibody are defined, based on the amino acid sequence of the heavy and light chains of the antibody, according to criteria well-known to a person skilled in the art. The CDRs of the antibodies according to the invention are more specifically determined according to the IMGT nomenclature.

Furthermore, functional antibody fragment denotes any fragment of the antibody conserving the ability to bind to the antigen and hence having the same affinity for the OprF protein of Pseudomonas aeruginosa than the original antibody. Such fragments can in particular be Fv, scFv, Fab, Fab′, F(ab′)2 fragments, nanobodies, etc. The antibody fragments according to the present invention can also comprise peptide sequences not belonging to the original antibody, corresponding for example to binding peptides between parts of the antibody, such as heavy chain and light chain parts, or to peptide tags, for example C-terminal, enabling for example their purification, their detection, etc., such as a polyhistidine tag and a c-myc tag, well-known to a person skilled in the art.

The expression “antibody fragment” also includes multivalent forms of antibody fragments, in particular the bi-, tri- or tetravalent forms of two, three or four fragments, particularly of scFv, such as diabodies, triabodies and tetrabodies.

The monoclonal antibody according to the invention can be of the bispecific type, or more generally have any multivalent form.

Single-chain variable fragments (scFv), fusion proteins between the variable region of the heavy chain VH and the variable region of the light chain VL, are particularly preferred within the scope of the invention. These scFv fragments can in particular comprise, between these variable regions, a binding peptide linking the heavy chain variable region and the light chain variable region. This binding peptide may be any conventional peptide in the scFv domain. It preferably comprises at least 5 amino acids, and preferably between 5 and 20 amino acids approximately. It can for example have the sequence GGGGSGGGGSGGGGS (SEQ ID NO: 11). Other examples of binding peptides that can be used according to the invention are described in the publication by Chen et al., 2013, Adv. Drug Deliv. Rev. 65(10): 1357-1369 (in particular in Table 3).

The binding peptide can connect the N-terminus of the heavy chain variable region and the C-terminus of the light chain variable region. Preferably, it connects the N-terminus of the light chain variable region and the C-terminus of the heavy chain variable region.

The scFv fragments can be produced from the complementary DNA (cDNA) coding for the variable region of the heavy chain VH and the cDNA coding for the variable region of the light chain VL, for example obtained from a hybridoma, a bacterium, an acellular system or any other recombinant protein production system producing the antibody according to the invention, according to conventional protein expression techniques.

More generally, the antibodies or antibody fragments according to the invention can be produced by genetic recombination or by chemical synthesis, or be isolated by purifying from a natural source, in particular from a hybridoma.

The antibodies or antibody fragments complying with the definition of the invention have a high affinity for the OprF protein of Pseudomonas aeruginosa. The dissociation constant of the binding of these antibodies or antibody fragments with the antigen can in particular be of the order of 200 nM. These antibodies furthermore have a high neutralizing power with respect to the bacterium in cellulo.

For simplification purposes, the monoclonal antibody according to the invention will be referred to in the present description as “antibody”, and the functional fragment of this antibody as “antibody fragment” or “fragment of this antibody”.

According to the present invention, a sequence having at least 80%, preferably at least 85%, preferably at least 90%, preferably at least 95%, preferably at least 96%, preferably at least 97%, preferably at least 98%, and preferentially at least 99%, identity with a reference sequence is a sequence having one or more variations with respect to this reference sequence, while providing the antibody or the antibody fragment with an affinity for the antigen, as for the reference sequence. These variations can be deletions, substitutions and/or insertions of one or more amino acids in the sequence.

The percentage identity corresponds to the percentage of identical amino acids between the compared sequences, obtained after optimal alignment of the two sequences. The optimal alignment of the sequences can be carried out in any conventional manner for a person skilled in the art, for example using the BLAST software. The percentage of identity is calculated by determining the number of positions for which the amino acid is identical between the two sequences, and by dividing it by the total number of positions in the sequence, the result being multiplied by 100.

When the sequence of the CDR of an antibody or antibody fragment according to the invention has a percentage identity less than 100% relative to one of the sequences listed above, in particular relative to one of the sequences SEQ ID NO: 1, SEQ ID NO: 2, SEQ ID NO: 3, or SEQ ID NO: 4, it can have insertions, deletions and/or substitutions relative to this reference sequence. In the case of a substitution, the substitution is preferably carried out by an amino acid from the same family as the original amino acid, for example of a basic residue such as arginine by another basic residue such as a lysine residue, of an acidic residue such as aspartate by another acidic residue such as glutamate, of a polar residue such as serine by another polar residue such as threonine, of an aliphatic residue such as leucine by another aliphatic residue such as isoleucine, etc.

Preferably, the antibody or antibody fragment according to the invention complies with one or more of the following features:
Xaa₁ is a threonine residue,
Xaa₃ is an arginine residue,
Xaa₅ is a glutamic acid residue,
Xaa₆ is an alanine residue,
and/or Xaa₁₃ is a valine residue.

Preferably, the antibody or antibody fragment according to the invention is such that in the sequence SEQ ID NO: 1, corresponding to the CDR of the heavy chain VH-CDR1, Xaa₃ is an arginine residue and in the sequence SEQ ID NO: 2, corresponding to the CDR of the heavy chain VH-CDR2, Xaa₆ is an alanine residue.

The antibody or antibody fragment according to the invention is furthermore preferably such that in the sequence SEQ ID NO: 1, corresponding to the CDR of the heavy chain VH-CDR1, Xaa₁ is a threonine residue, in the sequence SEQ ID NO: 2, corresponding to the CDR of the heavy chain VH-CDR2, Xaa₅ is a glutamic acid residue and in the sequence SEQ ID NO: 4, corresponding to the CDR of the light chain VL-CDR3, Xaa₁₃ is a valine residue.

Particularly preferred sequences according to the invention are the following sequences:

for VH-CDR1:
(SEQ ID NO: 5)
GYTFSRFG,
(SEQ ID NO: 6)
GYSFSSYG,
(SEQ ID NO: 7)
GYSFSTYG,
(SEQ ID NO: 8)
GYSFSRYG,
(SEQ ID NO: 9)
GYSFNTYG
or
(SEQ ID NO: 10)
GYSFSTFG;
for VH-CDR2:
(SEQ ID NO: 12)
INAETGKA,
(SEQ ID NO: 13)
INADTGKS,
(SEQ ID NO: 14)
INADTGKA
or
(SEQ ID NO: 15)
INAETGKS;
for VL-CDR1:
(SEQ ID NO: 16)
SSVTTNY,
(SEQ ID NO: 17)
SSVTTGY,
(SEQ ID NO: 18)
SSVNTNY,
(SEQ ID NO: 19)
SSVSTNY,
(SEQ ID NO: 20)
SSVATGF,
(SEQ ID NO: 21)
SSVSTSY,
(SEQ ID NO: 22)
SSVRTGY
or
(SEQ ID NO: 23)
SSVSTGY;
for VL-CDR2:
GTS
or
RTS;
for VL-CDR3:
(SEQ ID NO: 24)
QQGHSV,
(SEQ ID NO: 25)
QQGHTI,
(SEQ ID NO: 26)
QQGNTI
or
(SEQ ID NO: 27)
QQGHSI.

Specific antibodies or antibody fragments according to the invention are such that:
the complementarity determining regions (CDR) of the heavy chain variable region have the following respective amino acid sequences, or sequences having at least 80%, preferably at least 85%, preferably at least 90%, preferably at least 95%, preferably at least 96%, preferably at least 97%, preferably at least 98%, and preferentially at least 99%, identity with these sequences:

VH-CDR1:
(SEQ ID NO: 5)
GYTFSRFG
VH-CDR2:
(SEQ ID NO: 12)
INAETGKA
VH-CDR3:
VR

and/or the complementarity determining regions (CDR) of the light chain variable region have the following respective amino acid sequences, or sequences having at least 80%, preferably at least 85%, preferably at least 90%, preferably at least 95%, preferably at least 96%, preferably at least 97%, preferably at least 98%, and preferentially at least 99%, identity with these sequences:

VL-CDR1:
(SEQ ID NO: 16)
SSVTTNY
VL-CDR2:
GTS
VL-CDR3:
(SEQ ID NO: 24)
QQGHSV.

Other specific antibodies or antibody fragments according to the invention are such that:
the complementarity determining regions (CDR) of the heavy chain variable region have the following respective amino acid sequences, or sequences having at least 80%, preferably at least 85%, preferably at least 90%, preferably at least 95%, preferably at least 96%, preferably at least 97%, preferably at least 98%, and preferentially at least 99%, identity with these sequences:

VH-CDR1:
(SEQ ID NO: 6)
GYSFSSYG
VH-CDR2:
(SEQ ID NO: 13)
INADTGKS
VH-CDR3:
VR

and/or the complementarity determining regions (CDR) of the light chain variable region have the following respective amino acid sequences, or sequences having at least 80%, preferably at least 85%, preferably at least 90%, preferably at least 95%, preferably at least 96%, preferably at least 97%, preferably at least 98%, and preferentially at least 99%, identity with these sequences:

VL-CDR1:
(SEQ ID NO: 17)
SSVTTGY
or
(SEQ ID NO: 16)
SSVTTNY
VL-CDR2:
GTS
VL-CDR3:
(SEQ ID NO: 25)
QQGHTI
or
(SEQ ID NO: 26)
QQGNTI.

Other specific antibodies or antibody fragments according to the invention are such that:
the complementarity determining regions (CDR) of the heavy chain variable region have the following respective amino acid sequences, or sequences having at least 80%, preferably at least 85%, preferably at least 90%, preferably at least 95%, preferably at least 96%, preferably at least 97%, preferably at least 98%, and preferentially at least 99%, identity with these sequences:

VH-CDR1:
(SEQ ID NO: 7)
GYSFSTYG
or
(SEQ ID NO: 8)
GYSFSRYG
VH-CDR2:
(SEQ ID NO: 13)
INADTGKS
or
(SEQ ID NO: 14)
INADTGKA
VH-CDR3:
VR

and/or the complementarity determining regions (CDR) of the light chain variable region have the following respective amino acid sequences, or sequences having at least 80%, preferably at least 85%, preferably at least 90%, preferably at least 95%, preferably at least 96%, preferably at least 97%, preferably at least 98%, and preferentially at least 99%, identity with these sequences:

VL-CDR1:
(SEQ ID NO: 18)
SSVNTNY,
(SEQ ID NO: 17)
SSVTTGY
or
(SEQ ID NO: 16)
SSVTTNY
VL-CDR2:
GTS
VL-CDR3:
(SEQ ID NO: 25)
QQGHTI
or
(SEQ ID NO: 26)
QQGNTI.

Specific antibodies or antibody fragments according to the invention have CDRs of sequences indicated in Table 1 hereinafter, the sequence of VH-CDR3 being VR:

TABLE 1

| Anti- | | | | | |
|---|---|---|---|---|---|
| body | | | | | |
| or | | | | | |
| frag- | VH- | VH- | VL- | VL- | VL- |
| ment | CDR1 | CDR2 | CDR1 | CDR2 | CDR3 |
| R1 | GYTFSRFG | INAETGKA | SSVTTNY | GTS | QQGHSV |
| | (SEQ ID | (SEQ ID | (SEQ ID | | (SEQ ID |
| | NO: 5) | NO: 12) | NO: 16) | | NO: 24) |
| R2 | GYSFSSYG | INADTGKS | SSVTTGY | GTS | QQGHTI |
| | (SEQ ID | (SEQ ID | (SEQ ID | | (SEQ ID |
| | NO: 6) | NO: 13) | NO: 17) | | NO: 25) |
| R3 | GYSFSTYG | INADTGKS | SSVNTNY | GTS | QQGNTI |
| | (SEQ ID | (SEQ ID | (SEQ ID | | (SEQ ID |
| | NO: 7) | NO: 13) | NO: 18) | | NO: 26) |
| R4 | GYSFNTYG | INADTGKS | SSVSTNY | RTS | QQGNTI |
| | (SEQ ID | (SEQ ID | (SEQ ID | | (SEQ ID |
| | NO: 9) | NO: 13) | NO: 19) | | NO: 26) |
| R5 | GYSFSTFG | INADTGKS | SSVNTNY | GTS | QQGNTI |
| | (SEQ ID | (SEQ ID | (SEQ ID | | (SEQ ID |
| | NO: 10) | NO: 13) | NO: 18) | | NO: 26) |
| R6 | GYSFSTFG | INADTGKS | SSVATGF | GTS | QQGHSI |
| | (SEQ ID | (SEQ ID | (SEQ ID | | (SEQ ID |
| | NO: 10) | NO: 13) | NO: 20) | | NO: 27) |
| R7 | GYSFNTYG | INAETGKS | SSVSTSY | ATS | QQGHTI |
| | (SEQ ID | (SEQ ID | (SEQ ID | | (SEQ ID |
| | NO: 9) | NO: 15) | NO: 21) | | NO: 25) |
| R8 | GYSFSSYG | INADTGKS | SSVRTGY | GTS | QQGNTI |
| | (SEQ ID | (SEQ ID | (SEQ ID | | (SEQ ID |
| | NO: 6) | NO: 13) | NO: 22) | | NO: 26) |
| R9 | GYSFSTFG | INADTGKS | SSVATGF | GTS | QQGHSI |
| | (SEQ ID | (SEQ ID | (SEQ ID | | (SEQ ID |
| | NO: 10) | NO: 13) | NO: 20) | | NO: 27) |
| R10 | GYSFNTYG | INAETGKS | SSVNTNY | GTS | QQGNTI |
| | (SEQ ID | (SEQ ID | (SEQ ID | | (SEQ ID |
| | NO: 9) | NO: 15) | NO: 18) | | NO: 26) |
| R11 | GYSFSTYG | INADTGKS | SSVSTGY | ATS | QQGHSI |
| | (SEQ ID | (SEQ ID | (SEQ ID | | (SEQ ID |
| | NO: 7) | NO: 13) | NO: 23) | | NO: 27) |

Other specific antibodies or antibody fragments according to the invention have CDRs of sequences indicated in Table 2 hereinafter, the sequence of VH-CDR3 being VR:

TABLE 2

| Antibody | | | | | |
|---|---|---|---|---|---|
| or | | | | | |
| fragment | VH-CDR1 | VH-CDR2 | VL-CDR1 | VL-CDR2 | VL-CDR3 |
| P1 | GYSFSSYG | INADTGKS | SSVTTNY | GTS | QQGNTI |
| | (SEQ ID | (SEQ ID | (SEQ ID | | (SEQ ID |
| | NO: 6) | NO: 13) | NO: 16) | | NO: 26) |
| P2 | GYSFSSYG | INADTGKS | SSVTTGY | GTS | QQGNTI |
| | (SEQ ID | (SEQ ID | (SEQ ID | | (SEQ ID |
| | NO: 6) | NO: 13) | NO: 17) | | NO: 26) |
| P3 | GYSFSSYG | INADTGKS | SSVTTNY | GTS | QQGHTI |
| | (SEQ ID | (SEQ ID | (SEQ ID | | (SEQ ID |
| | NO: 6) | NO: 13) | NO: 16) | | NO: 25) |
| P4 | GYSFSRYG | INADTGKA | SSVTTNY | GTS | QQGNTI |
| | (SEQ ID | (SEQ ID | (SEQ ID | | (SEQ ID |
| | NO: 8) | NO: 14) | NO: 16) | | NO: 26) |
| P5 | GYSFSRYG | INADTGKA | SSVTTGY | GTS | QQGNTI |
| | (SEQ ID | (SEQ ID | (SEQ ID | | (SEQ ID |
| | NO: 8) | NO: 14) | NO: 17) | | NO: 26) |
| P6 | GYSFSRYG | INADTGKA | SSVTTNY | GTS | QQGHTI |
| | (SEQ ID | (SEQ ID | (SEQ ID | | (SEQ ID |
| | NO: 8) | NO: 14) | NO: 16) | | NO: 25) |
| P7 | GYSFSRYG | INADTGKA | SSVTTGY | GTS | QQGHTI |
| | (SEQ ID | (SEQ ID | (SEQ ID | | (SEQ ID |
| | NO: 8) | NO: 14) | NO: 17) | | NO: 25) |

A specific antibody or antibody fragment according to the invention comprises a pair of sequences chosen from the following pairs of sequences: SEQ ID NO: 28 and SEQ ID NO: 29, SEQ ID NO: 30 and SEQ ID NO: 31, SEQ ID NO: 32 and SEQ ID NO: 33, SEQ ID NO: 34 and SEQ ID NO: 35, SEQ ID NO: 36 and SEQ ID NO: 37, SEQ ID NO: 38 and SEQ ID NO: 39, SEQ ID NO: 40 and SEQ ID NO: 41, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, or a pair of sequences having at least 80%, preferably at least 85%, preferably at least 90%, preferably at least 95%, preferably at least 96%, preferably at least 97%, preferably at least 98%, and preferentially at least 99% , identity with one of these pairs of sequences. This means any pair of sequences wherein each of the sequences has at least 80%, preferably at least 85%, preferably at least 90%, preferably at least 95%, preferably at least 96%, preferably at least 97%, preferably at least 98%, and preferentially at least 99%, identity with respectively one of the sequences of a pair of sequences from the pairs of sequences listed above.

The sequences of a pair of sequences can be bound directly to one another, in particular by a peptide bond, or be bound to one another by a sequence of a binding peptide.

A specific antibody fragment according to the invention, in particular an scFv fragment, comprises, and preferably consists of, a sequence chosen from the sequences SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59 and SEQ ID NO: 60, or a sequence having at least 80%, preferably at least 85%, preferably at least 90%, preferably at least 95%, preferably at least 96%, preferably at least 97%, preferably at least 98%, and preferentially at least 99%, identity with one of these sequences.

In particular, the binding peptide of sequence (G₄S)₃ (SEQ ID NO: 11) comprised in these sequences can be replaced by any other binding peptide.

Preferably, the heavy chain variable region and the light chain variable region of the antibody or antibody fragment according to the invention are from the macaque (Macaca fascicularis). The same can apply for the heavy chain constant region and/or the light chain constant region. The macaque particularly has the advantage of very high homology of genetic sequences with humans. Otherwise, one or more of these regions can be from a transgenic animal.

Preferentially, the antibody or antibody fragment according to the invention does not have the amino acid sequence SEQ ID NO: 73. In particular it may not comprise a light chain variable region having the sequence SEQ ID NO: 74, or a light chain variable region CDR having the sequence SEQ ID NO: 75, i.e., a light chain variable region CDR of sequence SSVXaa₇TXaa₈Xaa₉ (SEQ ID NO: 3) wherein Xaa₇, Xaa₈ and Xaa₉ are as defined above, but Xaa₇ and Xaa₈ are not, simultaneously, an asparagine residue for Xaa₇ and a serine residue for Xaa₈.

The antibody or antibody fragment according to the invention can be a recombinant antibody or antibody fragment comprising the paratope of an antibody produced by a hybridoma, in particular from the macaque, and the constant regions of which have been modified so as to minimize the immunogenicity with respect to humans. For example, it is a chimeric antibody or antibody fragment or a humanized antibody or antibody fragment.

A chimeric antibody or antibody fragment denotes here, conventionally, an antibody or antibody fragment which contains a natural variable region derived from an antibody of a given species, in association with the constant regions of an antibody of a species which is heterologous to this species. Such antibodies can for example be prepared by genetic recombination.

The chimeric antibody or antibody fragment according to the invention is preferably such that the heavy chain constant region and/or the light chain constant region, if it includes any, are of human origin, the variable regions being for their part derived from the macaque.

A humanized antibody or antibody fragment comprises CDRs from a non-human mammalian antibody, preferably, according to the present invention, from macaque, and framework regions FR and C derived from human antibody. It is within the skills of a person skilled in the art to determine which modifications can be made to a given antibody to humanize it. For example, this humanization can be carried out by fusion with a heavy chain constant of sequence SEQ ID NO: 61 (human IgG1, G1m1,17 allotype).

The scope of the present invention also includes antibodies or antibody fragments which are modified, while retaining their affinity for the OprF protein of Pseudomonas aeruginosa, for example in order to optimize some of the effector functions thereof. The modifications can be made on an amino acid residue or a peptide bond. An example of such a modification is the binding of polyethylene glycol.

Another aspect of the invention relates to a nucleic acid molecule coding for a monoclonal antibody or functional fragment of said antibody according to the invention.

This nucleic acid molecule can for example have a sequence chosen from the sequences SEQ ID NO: 62 to SEQ ID NO: 72. These sequences code for antibody fragments according to the invention, wherein a binding peptide of sequence (G₄S)₃ (SEQ ID NO: 11) links the heavy chain variable region and the light chain variable region.

The invention also relates to an expression vector comprising a nucleic acid molecule according to the invention. This expression vector can be of any type known per se for use in genetic engineering, in particular a plasmid, a cosmid, a virus, a bacteriophage, containing the necessary elements for the transcription and translation of the sequence coding for the antibody or functional fragment of this antibody according to the invention.

The present invention also relates to a host cell comprising a nucleic acid molecule or an expression vector according to the invention. This host cell can equally well be a prokaryotic cell, in particular bacterial, particularly for the mass production of the antibody or functional fragment of this antibody, or a eukaryotic cell, which can be of lower or higher eucaryote, for example of yeast, invertebrates or mammals. In particular, the scope of the invention includes cell lines expressing, in a stable, inducible or constitutive manner, or else in a transient manner, an antibody or functional fragment of this antibody according to the invention.

The antibodies or antibody fragments according to the invention can be produced by any conventional method known to a person skilled in the art. They can particularly be obtained by genetic recombination or by chemical synthesis.

According to a specific implementation of the invention, a method for preparing an antibody or antibody fragment according to the invention comprises the culture of host cells according to the invention, i.e., comprising a nucleic acid molecule coding for an antibody or antibody fragment according to the invention, or an expression vector containing such a nucleic acid molecule, under conditions enabling the expression of said monoclonal antibody or functional fragment of said antibody, and the recovery of the antibody, or functional fragment of this antibody, thus produced.

Alternative methods for preparing an antibody or antibody fragment according to the invention also fall within the scope of the invention, in particular by inoculating a non-human mammal with the OprF antigen of Pseudomonas aeruginosa, optionally with a Freund's adjuvant, and screening the hybridomas producing an antibody having an affinity for this antigen, the antibodies or antibody fragments according to the invention being identified by analyzing the sequence thereof. For this purpose, the OprF protein used for inoculation is in the form of proteoliposomes, such as described in the publication by Maccarini et al. cited above.

The inoculation can be carried out by any route, in particular by subcutaneous, intramuscular, intravenous, intraperitoneal, etc., injection. One or more injections, several days apart, can be administered.

A method for preparing an antibody or antibody fragment according to a specific embodiment of the invention comprises successive steps of:
producing proteoliposomes containing the OprF protein of Pseudomonas aeruginosa. This step can be carried out by contacting, to form a reaction medium, an expression vector containing the coding sequence of porin OprF of Pseudomonas aeruginosa and a synthetic liposome, in particular of defined lipid composition, in the presence of an acellular protein synthesis system, this system, for example, being optionally obtained from a bacterial lysate or any other lysate obtained from yeasts, mammalian cells, wheat germ or any other biological source, this system enabling the transcription and simultaneous translation of the protein, for example according to the protocol described in the publication by Maccarini et al. cited above;
inoculating a non-human mammal, in particular of the species Macaca fascicularis, with said proteoliposomes;
constructing a bank of antibody or antibody fragments, particularly of scFv, from RNA extracted from B lymphocytes of said mammal,
screening said bank with respect to said proteoliposomes, by an expression technique, in particular phage display and enzyme-linked immunoadsorbent assay (ELISA) for example,
and selecting and recovering the clones which are reactive against the proteoliposome target.

According to the present invention, a clone is considered to be reactive against the proteoliposome target when the dissociation constant thereof, as measured by ELISA, with respect to this target, is less than or equal to 10 μM.

Preferentially, the method comprises, for the clones which are reactive with respect to the proteoliposomes thus selected, a step of isolating and verifying any redundancy thereof by sequencing.

The construction of a bank of antibodies or antibody fragments, particularly scFv, from RNA extracted from B lymphocytes of said mammal, can for example comprise amplification by reverse transcription and polymerase chain reaction (RT-PCR) of the messenger RNA (mRNA) coding for the variable domains VLk, VLλ and VH of the antibodies, construction of a bank of scFv by sequential cloning of the variable domains VLk, VLλ and VH in a phagemid vector, and encapsidation and amplification of the bank of scFv in phage, particularly in phage M13KO7 from the company Invitrogen®.

The method for preparing an antibody or functional antibody fragment according to the invention can comprise a step of humanizing an antibody or antibody fragment produced from the non-human mammal, by grafting at least one CDR sequence of this antibody or antibody fragment to a framework region FR of a human antibody.

It can furthermore comprise substitutions, insertions and/or deletions of one or more amino acids of an antibody or antibody fragment produced from the non-human mammal.

The antibody or antibody fragment according to the invention finds a particularly advantageous application for the treatment of bacterial infections, in particular Pseudomonas aeruginosa infections.

Thus, according to another aspect, the present invention relates to a pharmaceutical composition, in particular a vaccine composition, for combatting bacterial infections, in particular Pseudomonas aeruginosa infections, and particularly acute and chronic lung infections. This composition comprises a monoclonal antibody or functional fragment of said antibody according to the invention as an active substance, in a pharmaceutically acceptable vehicle.

The pharmaceutical composition according to the invention can have any dosage form suitable for administration to a mammal, in particular a dosage form suitable for oral or parenteral administration. In particular, it can be presented in a dosage form suitable for intravenous, intramuscular, intraperitoneal or subcutaneous injection, or for administration by the intranasal route or by inhalation.

The vehicle can consist of any conventional vehicle known per se, particularly in the field of vaccine compositions. It can in particular consist of an aqueous vehicle.

The pharmaceutical composition according to the invention can furthermore contain any conventional additive known per se, as well as optionally other active substances.

As additives that can be used in the pharmaceutical composition according to the invention, mention can be made of surfactants, in particular of polysorbate type, solvents or stabilizing agents, for example such as glycine, arginine or others, etc.

Another aspect of the invention relates to the use, for preventive or curative purposes, of a monoclonal antibody or functional fragment of this antibody as a medicament, and in particular for combatting bacterial infections, in particular Pseudomonas aeruginosa infections, particularly respiratory system infections, and more particularly lung infections, in particular acute and chronic lung infections.

This use comprises administering said antibody or antibody fragment, or a pharmaceutical composition containing same, to a mammal, in particular a human, in a therapeutically effective dose.

This administration can be performed by any route. It is preferably performed by the oral route or by the parenteral route, in particular by intravenous, intramuscular, intraperitoneal or subcutaneous injection, or by the intranasal route or by inhalation.

The antibody or antibody fragment according to the invention can be administered to the treated individual in a single dose, or in several doses, in particular administered several days apart.

The effective dose, the duration of administration and the number of administrations are dependent on the treated individual, in particular on its age, weight, symptoms, etc. Determining the exact treatment conditions is within the remit of the practitioner.

For example, a therapeutically effective dose of the antibody or antibody fragment according to the invention can be between 1 and 1000 mg, for a single-dose treatment.

The antibody or antibody fragment according to the invention can be used to treat any individual in need thereof, particularly any individual suffering from a bacterial infection, in particular a Pseudomonas aeruginosa infection, or, by way of prevention, any at-risk individual liable to contract such an infection, for example immunocompromised patients, patients with cystic fibrosis, patients under mechanical ventilation or severely burned patients, during hospitalization. Such a preventive treatment makes it possible to eliminate the risks of Pseudomonas aeruginosa infection considerably.

The present invention also relates to other uses of the antibody or antibody fragment according to the invention, for example for the detection, and optionally purification, of the OprF protein of Pseudomonas aeruginosa.

The present invention thus relates to the use of a monoclonal antibody or functional antibody fragment according to the invention for detecting, in vitro or ex vivo, the bacterium Pseudomonas aeruginosa in a biological fluid, in particular a body fluid obtained from an individual, in particular from a human or animal individual. It is thereby meant that the body fluid has been extracted from said individual.

This detection can be carried out by any conventional technique known per se by a person skilled in the art, for example by Western Blot, flow cytometry, surface plasma resonance, ELISA, etc.

Another aspect of the invention relates to a diagnostic kit for detecting the bacterium Pseudomonas aeruginosa in a biological fluid, in particular a body fluid from an individual, in particular a human or animal individual. This kit contains an antibody or functional antibody fragment according to the invention, and instructions for implementing a method for detecting, in vitro or ex vivo, the bacterium Pseudomonas aeruginosa in a body fluid obtained from an individual, by means of this monoclonal antibody or functional antibody fragment.

This kit can also comprise any conventional reagent known per se for the use of such a detection method.

The antibody or antibody fragment according to the invention can otherwise be used for preparing bi-specific antibodies.

### BRIEF DESCRIPTION OF THE DRAWINGS

The features and advantages of the invention will emerge more clearly in the light of the examples of implementation hereinafter, provided merely by way of illustration and not restriction of the invention, with reference to FIGS. 1 to 15, wherein:

FIG. 1 represents a graph showing the optical density at 450 nm, as a function of the serum dilution rate, for an ELISA test (affinity relative to the OprF protein of Pseudomonas aeruginosa) carried out on sera sampled from a macaque, on days 0, 24 and 38 after immunizing this macaque with proteoliposomes containing the OprF protein of Pseudomonas aeruginosa (OPRF D0, OPRF D24, OPRF D38) and on a serum sampled from a macaque, on day 38 after immunizing the macaque with bovine serum albumin (BSA D38).

FIG. 2 shows the sequence of 6 scFv fragments directed against the OprF protein of Pseudomonas aeruginosa according to the invention, wherein the sequences corresponding to the 6 CDRs, as well as the sequence of the binding peptide binding the heavy chain variable region to the light chain variable region, are underlined—in these sequences, the N-terminus is on the left, and the C-terminus is on the right, conventionally.

FIG. 3 shows the sequence of 5 other scFv fragments directed against the OprF protein of Pseudomonas aeruginosa according to the invention, wherein the sequences corresponding to the 6 CDRs, as well as the sequence of the binding peptide binding the heavy chain variable region to the light chain variable region, are underlined—in these sequences, the N-terminus is on the left, and the C-terminus is on the right, conventionally.

FIG. 4 represents a graph showing the optical density at 450 nm, as a function of the dilution rate, for ELISA tests (affinity relative to the OprF protein of Pseudomonas aeruginosa) carried out for different dilutions of an scFv fragment according to the invention (E2) and for bovine serum albumin (BSA) as a negative control.

FIG. 5 represents a graph showing the optical density at 450 nm, as a function of the dilution rate, for ELISA tests (affinity relative to the OprF protein of Pseudomonas aeruginosa) carried out for different dilutions of an scFv fragment according to the invention (E5) and for bovine serum albumin (BSA) as a negative control.

FIG. 6 represents a graph showing the optical density at 450 nm, as a function of the dilution rate, for ELISA tests (affinity relative to the OprF protein of Pseudomonas aeruginosa) carried out for different dilutions of an scFv fragment according to the invention (F8) and for bovine serum albumin (BSA) as a negative control.

FIG. 7 represents a graph showing the optical density at 450 nm, as a function of the dilution rate, for ELISA tests (affinity relative to the OprF protein of Pseudomonas aeruginosa) carried out for different dilutions of an scFv fragment according to the invention (G9) and for bovine serum albumin (BSA) as a negative control.

FIG. 8 represents a graph showing the optical density at 450 nm, as a function of the dilution rate, for ELISA tests (affinity relative to the OprF protein of Pseudomonas aeruginosa) carried out for different dilutions of an scFv fragment according to the invention (E3) and for bovine serum albumin (BSA) as a negative control.

FIG. 9 represents a graph showing the optical density at 450 nm, as a function of the dilution rate, for ELISA tests (affinity relative to the OprF protein of Pseudomonas aeruginosa) carried out for different dilutions of an scFv fragment according to the invention (E7) and for bovine serum albumin (BSA) as a negative control.

FIG. 10 represents a graph showing the optical density at 450 nm, as a function of the dilution rate, for ELISA tests (affinity relative to the OprF protein of Pseudomonas aeruginosa) carried out for different dilutions of an scFv fragment according to the invention (F10) and for bovine serum albumin (BSA) as a negative control.

FIG. 11 represents a graph showing the optical density at 450 nm, as a function of the dilution rate, for ELISA tests (affinity relative to the OprF protein of Pseudomonas aeruginosa) carried out for different dilutions of an scFv fragment according to the invention (F3) and for bovine serum albumin (BSA) as a negative control.

FIG. 12 represents a graph showing the optical density at 450 nm, as a function of the dilution rate, for ELISA tests (affinity relative to the OprF protein of Pseudomonas aeruginosa) carried out for different dilutions of an scFv fragment according to the invention (F4) and for bovine serum albumin (BSA) as a negative control.

FIG. 13 represents a graph showing the optical density at 450 nm, as a function of the dilution rate, for ELISA tests (affinity relative to the OprF protein of Pseudomonas aeruginosa) carried out for different dilutions of an scFv fragment according to the invention (A1) and for bovine serum albumin (BSA) as a negative control.

FIG. 14 represents a graph showing the optical density at 450 nm, as a function of the dilution rate, for ELISA tests (affinity relative to the OprF protein of Pseudomonas aeruginosa) carried out for different dilutions of an scFv fragment according to the invention (A8) and for bovine serum albumin (BSA) as a negative control.

FIG. 15 represents graphs showing the optical density (OD) at 450 nm as a function of the concentration, for ELISA tests (affinity relative to the OprF protein of Pseudomonas aeruginosa) carried out respectively on four scFv fragments according to the invention, named E7, F8, F10 and G9.

FIG. 16 shows a graph representing the value obtained by subtracting the absorbance at 680 nm from the absorbance at 490 nm (“A490-A680 nm”), during an in cellulo test for determining the neutralizing power, against the infection of macrophages (“Ma”) by Pseudomonas aeruginosa (“Pa”), of scFv fragments according to the invention (F8, G9, E7), by assaying the lactate dehydrogenase activity.

### DESCRIPTION OF THE PREFERRED EMBODIMENTS

### A/ Production of Proteoliposomes Containing the OprF Protein of Pseudomonas aeruginosa

### Construction of a Recombinant Vector Expressing OprF

The recombinant vector pIVEX2.4-OprF, wherein OprF comprises an N-terminal polyhistidine tag, is constructed by cloning the OprF gene amplified from genomic DNA of Pseudomonas aeruginosa amplified by polymerase chain reaction (PCR), by means of the following primers:

Sense
(SEQ ID NO: 76)
5′-GGAATTCCATATGAAACTGAAGAACACCTTAG-3′
Antisense
(SEQ ID NO: 77)
5′-TAGAAGCTGAAGCCAAGTAACTCGAGTAACGC-3′

in the expression vector pIVEX2.4d (Roche Diagnostics).

For this purpose, 30 PCR cycles are implemented using a high-fidelity DNA polymerase. The PCR product thus obtained is then purified by means of the QIAquick gel kit (Qiagen) then digested with the restriction enzymes Ndel, Xhol (Roche Diagnostics), purified once again then bound by means of the Rapid DNA ligation kit (Roche Diagnostics) in the plasmid vector pIVEX2.4d (Roche Diagnostics) previously digested by the enzymes Ndel and Xhol. The resulting recombinant plasmid pIVEX2.4-OprF is verified by sequencing (LGC Genomics) in order to validate the insertion of the gene coding for OprF in phase with the polyhistidine tag of the vector pIVEX2.4d.

### Liposome Preparation

Liposomes are prepared by drying a lipid composition previously solubilized in chloroform, for the following different lipid compositions (LC):
Lipid Composition 1 (LC1): cholesterol, 1,2-dioleoyl-sn-glycero-3-phosphocholine (DOPC), 1,2-dioleoyl-sn-glycero-3-phosphoethanolamine (DOPE), 1,2-dimyristoyl-sn-glycero-3-phosphate (sodium salt) (DMPA), molar ratio [2-4-2-2];
LC1′: LC1+1 mg/mL monophosphoryl lipid A (MPLA);
LC2: 1-palmitoyl-2-oleoyl-sn-glycero-3-phosphoethanolamine (POPE), 1-palmitoyl-2-oleoyl-sn-glycero-3-phospho-(1′-rac-glycerol) (sodium salt) (POPG), E. coli cardiolipin (CL), molar ratio [6-2-2];
LC2′: LC2+1 mg/mL MPLA;
LC3: POPE, POPG, E. coli CL, DMPA, molar ratio [6-2-1-1];
LC 3′: LC 3+1 mg/mL MPLA (Avanti Polar Lipids).

Drying is carried out by evaporation under nitrogen. The residual traces of chloroform are removed using a vacuum pump. The lipid film is then hydrated in 500 μl of a Tris solution (50 mM, pH 7.5) by vortexing, then subjected to 4 freezing/thawing cycles in liquid nitrogen. The lipid mixture is extruded using an extruder (Avanti Polar Lipids) to produce liposomes of an average size of approximately 200 nm. The liposomes thus obtained are stored at 4° C.

### Production and Purification of Proteoliposomes Containing OprF in Acellular System

The OprF membrane protein of Pseudomonas aeruginosa is synthesized in the presence of the different liposome compositions (LC1, or LC2, or LC3 or LC1′, or LC2′ or LC3′) using the RTS 500 ProteoMaster E. coli HY acellular protein synthesis kit, from Biotechrabbit. For this purpose, the recombinant plasmid pIVEX2.4-OprF containing the gene coding for the OprF protein fused with a polyhistidine tag (6xHis) is added at a concentration of 15 μg/ml to the cellular lysate of the kit in the presence of a quantity of 1 to 4 mg/ml of liposomes from one of the 6 lipid compositions LC1 to LC3′. Proteoliposomes containing the OprF recombinant protein are produced at 25° C. for 16 h under stirring (300 rpm) in a ratio of 1:30 of reaction volume/container volume. The resulting recombinant proteoliposomes are then purified in 2 steps:
firstly in a sucrose gradient of 0-40% in 50 mM pH 7.5 Tris buffer whereon the reaction mixture is deposited on the top of the gradient then centrifuged at 287.660 g for 2 h using a TH-641 rotor. Fractions of 1 ml are collected from the top of the gradient and analyzed by Western Blot using an anti-histidine antibody coupled with HRP horseradish peroxidase (Sigma),
then, 1 ml of a 50 mM pH 7.5 Tris buffer is added to each fraction containing the proteoliposomes containing OprF, and the solution is centrifuged at 30,000 g for 30 min at 4° C., to form a proteoliposome pellet. The pellet is washed twice for 30 min at 4° C. with a 5M NaCl solution and then resuspended in a 50 mM pH 7.5 Tris solution at the desired concentration. The purity of the samples is analyzed on an SDS-PAGE gel stained with Coomassie blue. Proteoliposomes containing the OprF protein of Pseudomonas aeruginosa are obtained.

### B/ Preparation and Screening of a Bank of scFv Fragments

### Immunization of Animals

scFv fragments targeted against the Pseudomonas aeruginosa OprF bacterial membrane antigenic target are obtained by an immunization with the proteoliposomes obtained from the lipid composition LC1, performed on days D0, D14, D28 and D50, of a macaque (Macaca fascicularis). The macaque is kept under sterile conditions in the presence of another similar animal and with no other species. Before the first injection, a blood test is performed to ascertain the physiological status of the animal. 100 μg of the composition LC1 in sterile phosphate buffered saline (PBS), mixed 50/50 with a Freund's adjuvant (complete for the first injection, incomplete for the subsequent injections) are injected in the animal subcutaneously at 2 points (at a rate of 250 μl per point) in the animal's scapular area, according to the following profile: administrations on days 0, 14, 28, 50.

The immune response is analyzed by immunoenzyme assay (ELISA) on sera sampled on days D0, D24, D38 to perform a titration of the antibodies targeted against the OprF membrane antigenic target. Bone marrow samples are taken after the final injection (D50) on D53, D60, D67 and D74 on the anesthetized animal.

### Serum Titration

The post-immunization humoral response is analyzed by the indirect ELISA method using a series of dilutions of the pre-immune and immune sera (dilutions of 100, 1000, 10,000, 1,000,000, 10,000,000 and 100,000,000) following the protocol described in the book “Phage Display”, Methods and Protocols, Springer Protocols, “Construction of Macaque Immune Libraries” chapter, Arnaud Avril et al., Methods Mol Biol. 2018; 1701: 83-112. doi: 10.1007/978-1-4939-7447-4_5. Briefly, the OprF membrane antigen in proteoliposome form or a negative control such as bovine serum albumin (BSA) is first deposited at the bottom of the ELISA plates and incubated for 16 h at 4° C. After a saturation step (2% of dried milk resuspended in 200 μl of PBS phosphate buffered saline), each diluted serum (1:100 initially then 1:10, in PBS/Tween® 0.05%/BSA 0.5%) is then tested in parallel against the OprF antigen or the negative control (BSA) for 2 h at 37° C. The specific antibodies against OprF are then detected using a secondary anti-macaque Fc antibody conjugated with HRP horseradish peroxidase and by adding tetramethylbenzidine (TMB) until a color appears in the wells. The results are analyzed by reading the optical density at 450 nm.

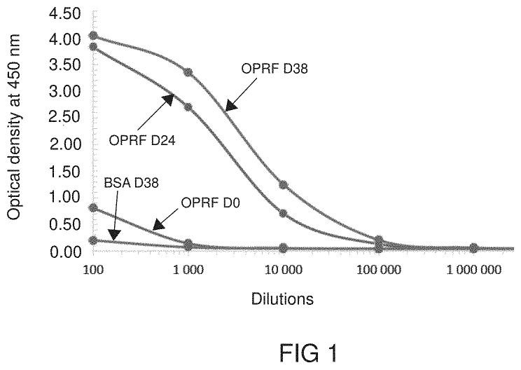

The results obtained are shown in FIG. 1, for the sera sampled on days D0, D24 and D38 post-immunization with the proteoliposomes containing OprF and for the sera obtained on day D38 for the immunization with BSA.

On day D38, a titer of 1:200000 is observed, which is compatible with the remainder of the method.

### Bone Marrow Samples and B Lymphocyte Isolation

Bone marrow samples are taken from the anesthetized animal. The samples are taken at the trochanteric fossa of the femur and at the tubercle of the humerus, by means of a Mallarme trocar. Each sample is collected in a 50 ml Falcon® tube containing a 10-15% citrate solution. Approximately 5 ml of sample are obtained on days D53, D60, D67 and D74. Each sample is then centrifuged for 10 min at 500 g (1500 rpm) at 4° C. The supernatant is removed and placed in a cryotube, then stored at −20° C. The total RNA of each of the bone marrows is then extracted with the Trizol/Chloroform technique and quantified by reading the optical density (OD) at 260 nm and 280 nm using a spectrophotometer.

The results obtained are shown in Table 3 hereinafter.

TABLE 3

| | OD at | OD at | OD 260 | [RNA] |
|---|---|---|---|---|
| Day | 260 nm | 280 nm | nm/280 nm | μg/ml |
| D53 | 6.239 | 3.390 | 1.840 | 249.557 |
| D60 | 16.215 | 8.446 | 1.920 | 648.609 |
| D67 | 3.460 | 1.921 | 1.801 | 138.395 |
| D74 | 5.313 | 2.911 | 1.825 | 212.535 |

### RT-PCR Amplification of the RNA Coding for the Variable Parts VLκ, VLλ and VH

The messenger RNA (mRNA) coding for the variable domains of the heavy and light chains G and κ/λ for each of the bone marrow samples are retro-amplified to obtain a bank of complementary DNA (cDNA) using specific primers, described in the publication by Avril et al., 2018, Methods Mol. Biol., 1701: 83-112.

The amplification quality is controlled by agarose gel electrophoresis.

The PCR products amplified from the cDNA bank obtained on days D53 to D74 are cloned in the plasmid pGemT (Promega), according to the supplier's protocol, in order to obtain a bank of secured clones.

### Construction of a Bank of scFv Fragments

From the DNA obtained on days D53 and D60, 2 banks (one for each day) are constructed by sequential cloning by inserting, according to the supplier's protocol, first the VL fragments in the phagemid vector pTh1 (Addgene) then the VH fragments, to obtain a construction in the format VH-[(G₄S)×3 (SEQ ID NO: 11)]-VL-6xHistidine-EQKLISEEDL (SEQ ID NO: MM), wherein the VH and VL fragments are bound by the binding peptide GGGGSGGGGSGGGGS (SEQ ID NO: 11) binding the C-terminus of the VH fragment to the N-terminus of the VL fragment, and comprising at its C-terminus a polyhistidine tag (SEQ ID NO: 79) and a c-myc tag (SEQ ID NO: 78).

For the DNA obtained on day D53, a bank of 1.5·10⁷ CFU (75% full size inserts) is obtained. For the DNA obtained on the day D60, a bank of 1·10⁷ CFU (100% full size inserts) is obtained.

### Screening of scFv Fragment Banks with the Phage Display Technique—Identification of Fragments Having an Affinity for the OprF Protein of Pseudomonas aeruginosa

The bank is encapsidated and amplified in phage M13Ko7 (Nebb), according to the supplier's protocol.

The scFv banks contained in the phagemids are subjected to 4 rounds of selection against the proteoliposomes containing the OprF membrane antigen fixed on 96-well plates.

The screening protocol is as follows: a microtitration plate is coated overnight with the targeted antigen at a concentration of 10 μg/ml in PBS at 4° C. Then, the plate is blocked with 3% BSA in PBS for 2 h at 37° C.; after washing, the bank is incubated for 2 further hours at 37° C. During the first round, the plate is washed twice using PBS containing 0.1% Tween® 20 with a 5-min interval between each wash. Finally, the plate is washed, rinsed with sterile PBS and the phage is eluted with trypsin (10 mg/ml in PBS) for 30 min at 37° C. The eluted phages are used for Escherichia coli infection (SURE strain, Stratagene, cultured in an SB (Super Broth) medium supplemented with tetracycline (10 μg/ml) and carbenicillin (50 μg/ml). For the production of new phage particles, the infected strain is co-infected with an auxiliary phage and cultured overnight at 30° C. in an SB medium supplemented with tetracycline (10 μg/mL), carbenicillin (50 μg/mL) and kanamycin (70 μg/mL). The phage particles are precipitated using PEG/NaCl (4% (w/v) PEG-8000, 3% (w/v) NaCl) and used for the next cycle. The second round is carried out as described above. The infected strains from the third round are cultured on SB media in Petri dishes and are used for the screening.

After each round, only the phages having interacted with OprF are eluted. The reactivity of the phages after each round of selection against the OprF target is tested by ELISA assay. The phages show a 30-fold signal increase between the first selection round and the 4^{th} round, indicating an enrichment of the scFvs which are reactive against OprF.

96 clones isolated from the second, third and fourth selection rounds are collected and used to produce soluble scFvs. From these clones, 57 positive clones are selected and 15 of these are retained. An analysis of the nucleotide and peptide sequence of the 57 clones is carried out to determine the potential redundancy of some sequences. 43 sequences are identified as non-redundant and non-recombined. Of these 43 sequences, 11 are produced in Escherichia coli bacteria, according to the following protocol: the phagemid DNA isolated after the selection process is used to transform the non-suppressive E. Coli strain such that it expresses the soluble scFv fragment. Single colonies of transformants selected at random are used to inoculate 5 ml of SB medium supplemented with carbenicillin. The cultures are incubated overnight at 37° C. under vigorous stirring (250 rpm). 500 ml of SB medium supplemented with carbenicillin are then inoculated with 500 μl of each culture. The cultures are cultured at 30° C. until the optical density at 600 nm reaches 1.5. IPTG (1 mM) is then added overnight to induce genic expression at 22° C. The cells are collected by centrifugation at 2500 g for 15 min at 4° C. The scFvs are extracted with polymyxin B sulfate and purified on a nickel column (Ni-NTA column, Qiagen) according to the manufacturer's instructions, then dialyzed against PBS.

The corresponding scFvs produced are then purified to confirm their affinity with respect to the OprF target with the ELISA method.

These 11 scFv fragments comprise the sequences indicated in Table 4 hereinafter.

TABLE 4

| scFv | Amino acid sequence |
|---|---|
| A1 | SEQ ID NO: 50 |
| A8 | SEQ ID NO: 51 |
| E2 | SEQ ID NO: 52 |
| E3 | SEQ ID NO: 53 |
| E5 | SEQ ID NO: 54 |
| E7 | SEQ ID NO: 55 |
| F3 | SEQ ID NO: 56 |
| F4 | SEQ ID NO: 57 |
| F8 | SEQ ID NO: 58 |
| F10 | SEQ ID NO: 59 |
| G9 | SEQ ID NO: 60 |

These sequences are prolonged therein, at their C-terminus, by a polyhistidine tag (SEQ ID NO: 79) and a c-myc tag (SEQ ID NO: 78).

These scFv fragments all comprise:
a heavy chain variable region having the three complementarity determining regions (CDR) having the following amino acid sequences:

VH-CDR1: GYXaa₁FXaa₂Xaa₃Xaa₄G (SEQ ID NO: 1) wherein Xaa₁ is a threonine residue or a serine residue, Xaa₂ is a serine residue or an asparagine residue, Xaa₃ is an arginine residue, a serine residue or a threonine residue and Xaa₄ is a phenylalanine residue or a tyrosine residue,

VH-CDR2: INAXaa₅TGKXaa₆ (SEQ ID NO: 2) wherein Xaa₅ is a glutamic acid residue or an aspartic acid residue and Xaa₆ is an alanine residue or a serine residue,

VH-CDR3: VR,
and a light chain variable region having the three CDRs having the following amino acid sequences:

VL-CDR1: SSVXaa₇TXaa₈Xaa₉ (SEQ ID NO: 3) wherein Xaa₇ is a threonine residue, an asparagine residue, a serine residue, an alanine residue or an arginine residue, Xaa₈ is an asparagine residue, a glycine residue or a serine residue and Xaa₉ is a tyrosine residue or a phenylalanine residue,

VL-CDR2: Xaa₁₀TS wherein Xaa₁₀ is a glycine residue, an arginine residue or an alanine residue,

VL-CDR3: QQGXaa₁₁Xaa₁₂Xaa₁₃ (SEQ ID NO: 4) wherein Xaa₁₁ is a histidine residue or an asparagine residue, Xaa₁₂ is a serine residue or a threonine residue and Xaa₁₃ is a valine residue or an isoleucine residue.

These scFv fragments are all according to the present invention.

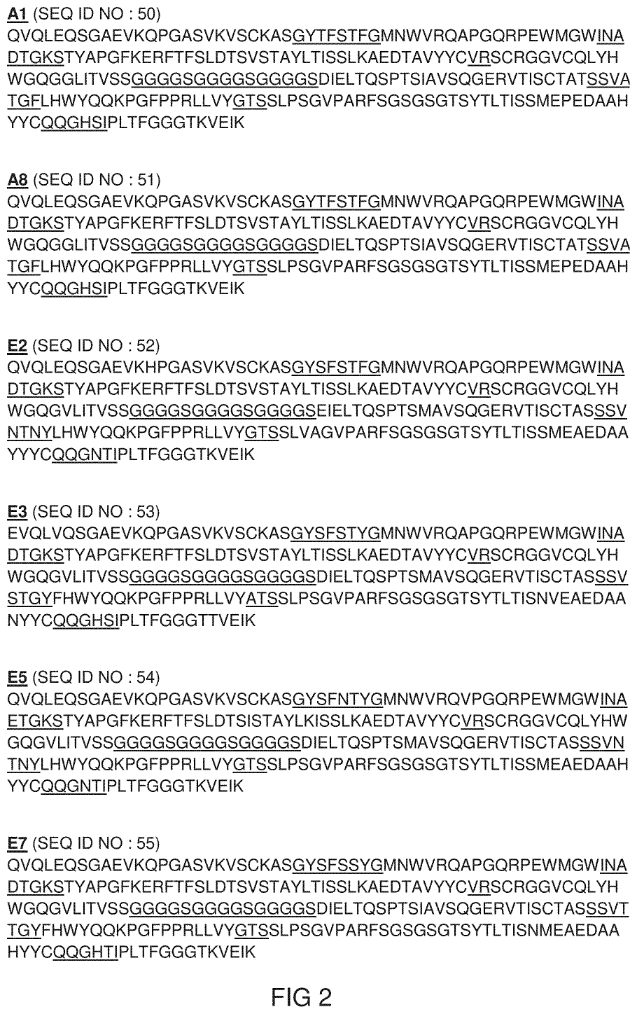

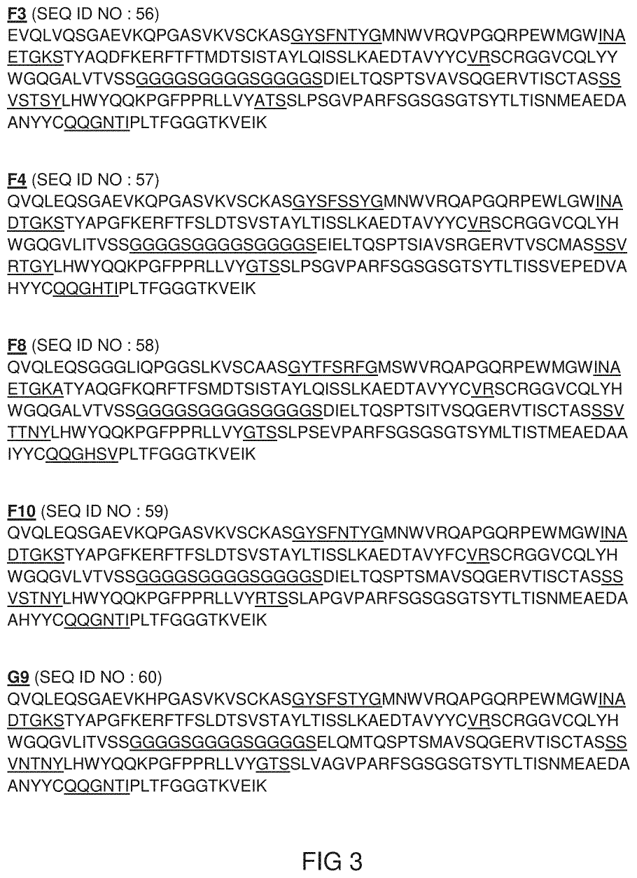

Each of the sequences of these scFv fragments is shown in FIG. 2 for A1, A8, E2, E3, E5, E7 and in FIG. 3 for F3, F4, F8, F10, G9. The CDR sequences are underlined therein. From left to right, the successive sequences of the CDRs of the heavy chain variable region are thus visible (VH-CDR1, followed by VH-CDR2, followed by VH-CDR3), followed by the light chain variable region (VL-CDR1, followed by VL-CDR2, followed by VL-CDR3). The binding peptide sequence is also underlined, between the three CDR triplets.

### C/ Analysis of the Affinity of scFv Fragments According to the Invention for the OprF Protein of Pseudomonas aeruginosa

The 11 scFv fragments produced above are purified.

The following quantities of each scFv fragment according to the invention are obtained: 0.346 mg/ml E2, 0.401 mg/ml E3, 0.559 mg/ml E5, 0.453 mg/ml E7, 0.387 mg/ml F4, 0.436 mg/ml F3, 0.333 mg/ml F8, 0.403 mg/ml F10, 0.570 mg/ml G9, 0.385 mg/ml A1 and 0.626 mg/ml A8.

An ELISA assay is carried out on MaxiSorp® plates, to confirm the affinity of these scFv fragments with respect to the OprF target in proteoliposome form, as follows.

The plate is saturated with 2.5% of dried milk resuspended in 200 μl of PBS phosphate buffered saline.

The scFv fragments are incubated at different dilution rates: 1:20, 1:40, 1:80, 1:160, 1:320, 1:640, 1:1280, in PBS/Tween®-20 0.05%/BSA 0.5%. Detection is performed using an anti-c-myc tag secondary antibody coupled with horseradish peroxidase. The optical density at 450 nm is recorded.

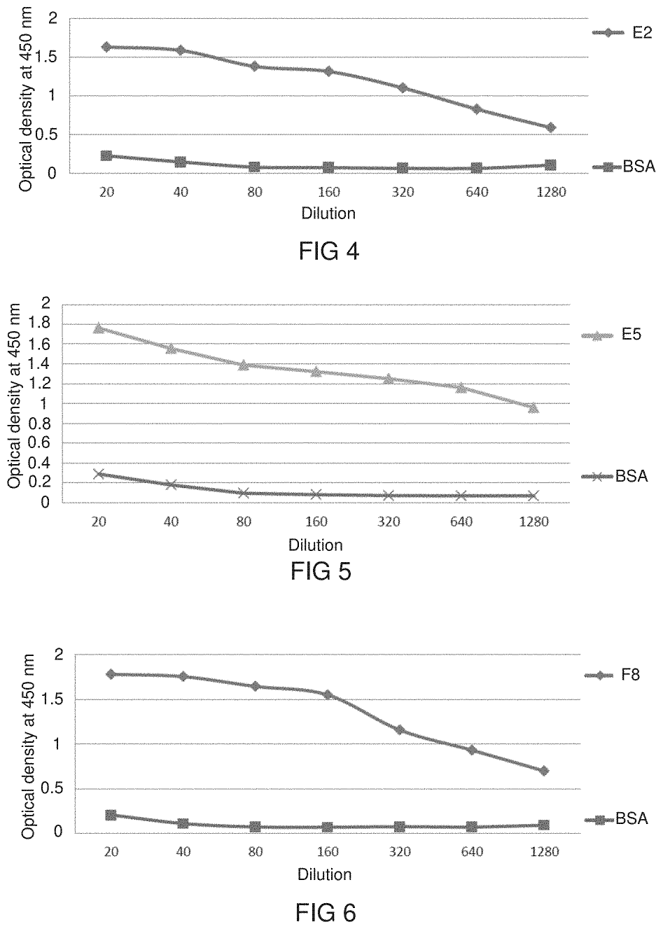

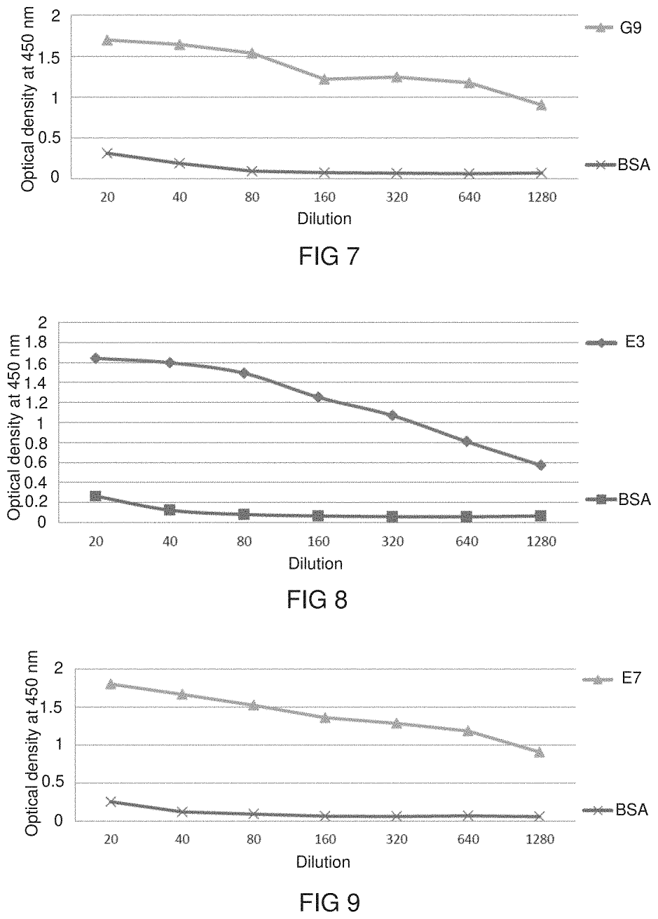

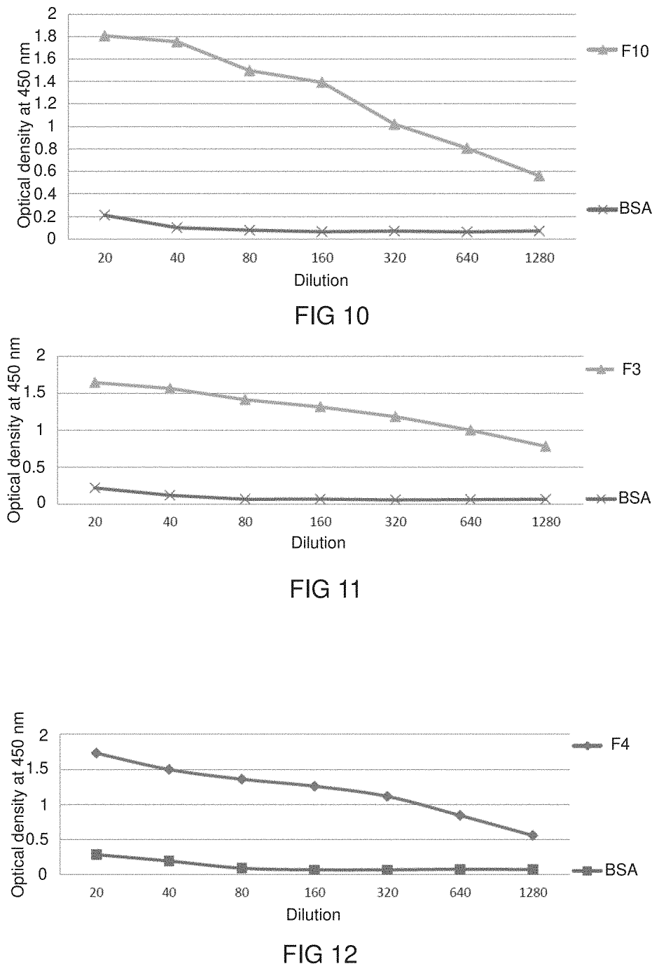

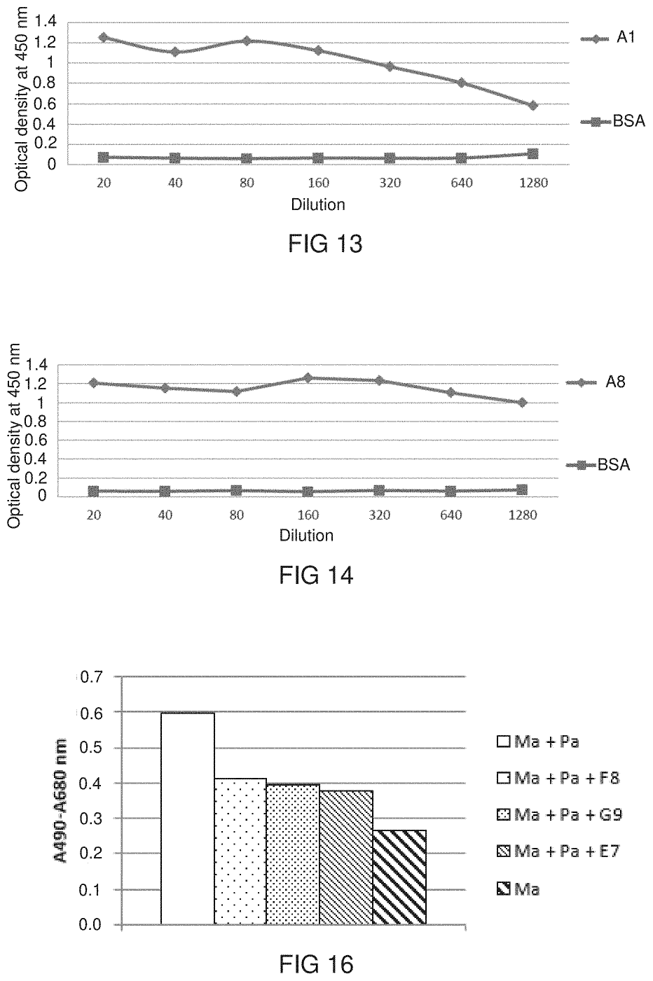

The results obtained, for the fragment according to the invention and for a negative control (BSA) are shown in FIG. 4 for E2, FIG. 5 for E5, FIG. 6 for F8, FIG. 7 for G9, FIG. 8 for E3, FIG. 9 for E7, FIG. 10 for F10, FIG. 11 for F3, FIG. 12 for F4, FIG. 13 for A1 and FIG. 14 for A8.

It can be seen that all of the scFv fragments according to the invention have a high affinity for the OprF protein of Pseudomonas aeruginosa.

By way of example, the dissociation constant Kd is determined for the scFv fragments E7, F8, F10 and G9, with an ELISA method.

For this purpose, 100 μl of OprF proteoliposomes (containing an OprF concentration of 1 μg/ml) contained in fixation buffer (0.1 M sodium carbonate, 0.1 M sodium bicarbonate) are fixed at the bottom of the wells of a 96-well plate (Thermo Scientific®) overnight at 4° C. and under stirring. The wells are then blocked for 1 h at 21° C. with 100 μl of TBS Tween (TBST) buffer containing 5% milk. After washing the wells with 100 μl of TBST buffer, 100 μl of scFv fragment (E7, F8, F10 or G9), at 1:50; 1:200; 1:400; 1:800; 1:1600 and 1:3200 dilutions in TBST buffer, are added to the corresponding wells and incubated for 1 h at 37° C. under stirring. After washing the wells, 100 μl of anti-c-myc-Peroxidase antibody (Roche) (1:10000 dilution in TBST buffer containing 5% milk) is added to the wells and incubated for 1 h at 37° C. under stirring. After washing the wells 3 times, 50 μl of TMB is added to the wells and the plate is incubated at ambient temperature and protected from light for approximately 15 min. 50 μl of 1 M HCl is then added and the absorbance of each well is measured at 450 nm. These absorbance data are then analyzed using the GraphPad Prism software (non-linear regression analysis, “one site-specific binding” equation) in order to calculate the dissociation constant Kd of each scFv fragment tested. By way of comparison, a measurement is also made on the buffer only.

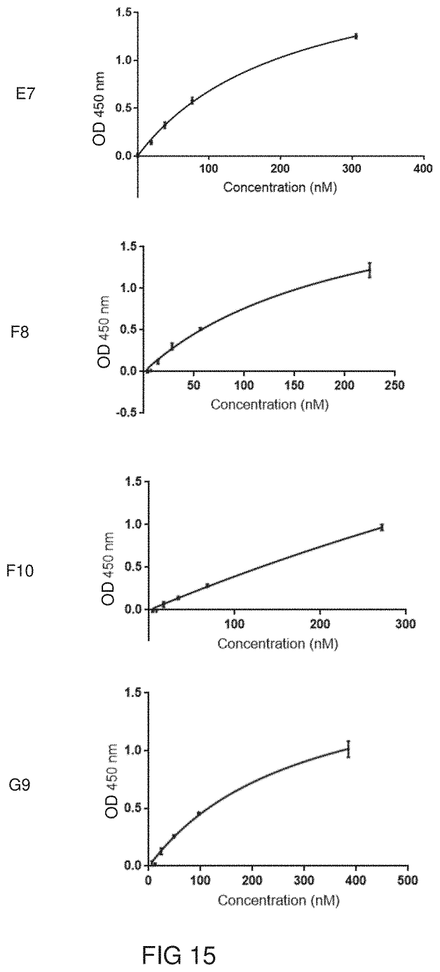

The results obtained, for each of the fragments E7, F8, F10 and G9, are shown in FIG. 15.

The values of the dissociation constants Kd thus determined are specified in Table 5 hereinafter.

TABLE 5

| scFv fragment | E7 | F8 | F10 | G9 |
|---|---|---|---|---|
| Kd (nM) | 213.4 | 211.4 | 1493 | 295.3 |

These results show a very good affinity of the fragments according to the invention for the proteoliposome target containing the OprF protein of Pseudomonas aeruginosa.

### D/ Determination of the Neutralizing Power of scFv Fragments According to the Invention in cellulo

The fragments E7, F8 and G9 were tested in this experiment, to determine their neutralizing power with respect to macrophage infection with Pseudomonas aeruginosa (CHA strain) at an MOI (multiplicity of infection) of 10.

The following protocol was implemented:
Differentiation of THP-1 cells (human monocytic line) into macrophages following the addition of phorbol myristate acetate (PMA): addition of 15 μl of PMA (0.1 mg/ml stock solution) to 10 ml of THP-1 cells suspended in RPMI medium containing 10% decomplemented fetal calf serum (dFCS) (440,000 cells/ml); incubation of the culture dish T25 in an oven at 37° C. (atmosphere containing 5% CO₂) for at least 48 h in order to enable the differentiation of the THP-1 cells into adherent macrophages;
Culture and preparation of P. aeruginosa (CHA strain): initiation of a culture of P. aeruginosa bacteria (CHA strain) from a small quantity of bacterial glycerol stock placed in 10 ml of LB culture medium; incubation of the bacterial culture at 37° C. under stirring overnight; dilution of the culture in LB medium until an optical density measurement at 600 nm equal to 0.5 in 1 ml of culture (6×10⁸ CFU/ml) is obtained; centrifugation at 4000 g for 5 min; removal of the supernatant and resuspension of the pellet in 1 ml of RPMI—10% dFCS culture medium; 2^{nd} centrifugation at 4000 g for 5 min; removal of the supernatant and resuspension of the pellet in 1 ml of RPMI—10% dFCS culture medium; dilution in the same medium in order to obtain a suspension containing 15,000,000 bacteria/ml; incubation at 37° C. for 2 h of: 35 μl of the P. aeruginosa suspension+35 μl of scFv E7 in PBS (0.453 mg/ml), 35 μl of the P. aeruginosa suspension+35 μl of scFv F8 in PBS (0.333 mg/ml), 35 μl of the P. aeruginosa suspension+35 μl of scFv G9 in PBS (0.570 mg/ml), 35 μl of the P. aeruginosa suspension;
Preparation of the macrophages: removal of the culture supernatant; addition of 2 ml of Versene in order to detach the adherent cells; after detaching the cell lawn, addition of 2 ml of medium and recovery of the cells; centrifugation at 400 g for 5 min; resuspension of the cell pellet in 2 ml of medium and enumeration of the cells; filling of the cell suspension in the wells of a 96-well plate so as to obtain 15,000 cells/well and addition of the necessary quantity of RPMI-10% dFCS medium in order to obtain the final volumes specified in Table 6 hereinafter:

TABLE 6
M represents macrophages

| | 1 | 2 | 3 | 4 | 5 |
|---|---|---|---|---|---|
| A | 90 μl of | 90 μl of | 90 μl of | 100 μl of | 100 μl of |
| | medium + M | medium + M | medium | medium | medium |
| B | 90 μl of | 90 μl of | 90 μl of | 100 μl of | 100 μl of |
| | medium + M | medium + M | medium | medium | medium |
| C | 90 μl of | 90 μl of | 90 μl of | 100 μl of | 100 μl of |
| | medium + M | medium + M | medium | medium | medium |
| D | 80 μl of | 80 μl of | 80 μl of | 80 μl of | |
| | medium + M | medium + M | medium + M | medium + M | |
| E | 80 μl of | 80 μl of | 80 μl of | 80 μl of | |
| | medium + M | medium + M | medium + M | medium + M | |
| F | 80 μl of | 80 μl of | 80 μl of | 80 μl of | |
| | medium + M | medium + M | medium + M | medium + M | |

Completion of the cytotoxicity test for quantifying the release of cellular LDH (Lactate Dehydrogenase) in the culture medium, according to the protocol defined in the instructions of the lactate dehydrogenase (LDH) assay kit, “Pierce® LDH cytotoxicity assay kit”: addition of 10 μl of sterile PBS to wells No. 1 to 5 of rows A, B, C and to wells No. 1 of rows D, E, F; addition of 10 μl of P. aeruginosa (Pa) to wells No. 3 of rows A, B, C and to wells No. 1 of rows D, E, F; addition of 20 μl of the mixture: Pa+F8 to wells No. 2 of rows D, E, F, Pa+G9 to wells No. 3 of rows D, E, F, Pa+E7 to wells No. 4 of rows D, E, F; addition of 10 μl of ultrapure sterile water to wells No. 1 and 3 of rows A, B, C; incubation of the plate in an oven at 37° C. (atmosphere containing 5% CO₂) for 16 h; addition of 10 μl of lysis buffer (10×) to wells No. 2 and 5 of rows A, B, C; incubation for 45 min in an oven at 37° C. (atmosphere containing 5% CO₂); centrifugation of the plate at 250 g for 3 min; transfer of 50 μl from each well into a new 96-well plate; addition to each well of 50 μl of the reaction mixture; incubation of the plate at ambient temperature and protected from light for 30 min; addition to each well of 50 μl of the stop solution; measurement of the absorbance of each well at 490 and 680 nm; subtraction of the absorbance values: A_{490 nm}-A_{680 nm}.

The results obtained are shown in FIG. 16. A reduction of approximately ⅔ in the cytotoxicity of P. aeruginosa with respect to the macrophages is observed in the presence of the ScFv fragments according to the invention. This demonstrates a neutralizing action of these fragments against P. aeruginosa.

### E/ Analysis of the Proteoliposomes Containing the OprF Protein of Pseudomonas aeruginosa Having Been Used to Obtain ScFvs According to the Invention

The proteoliposomes obtained in the experiment described in A/ above were subjected to the following analyses.

### E.1/ Materials and Methods

Digestion with trypsin—The OprF proteoliposomes (LC1) purified by centrifugation in sucrose gradient were proteolyzed using a trypsin:protein mass ratio of 1:10 at ambient temperature (RT). The samples were retrieved at different times and loaded on an SDS-PAGE gel for a subsequent Western Blot analysis.

Negative staining electron microscopy—The samples were prepared using the negative staining on grid (SOG) technique. 10 μl of OprF proteoliposomes (LC1, [OprF]: 0.1 mg/mL) or 10 μl of liposomes (4 mg/mL) incubated in the reaction mixture without DNA (=negative control) were added to a glow discharge grid coated with a carbon film for 3 min and the grid was stained with 50 μl of phosphotungstite acid (PTA, 1% in distilled water) for 2 min. The excess solution was absorbed by filter paper and the grid was air-dried. The images were taken under low-dose conditions (<10 e−/Å2) with defocus values between 1.2 and 2.5 μm on a Tecnai 12 LaB6 electron microscope at an acceleration voltage of 120 kV using the CCD Gatan Orius® 1000 camera. The mean pore size was determined using the open source image processing program ImageJ.

AFM tip functionalization—The golden tips (NPG-10, Bruker Nano AXS) were coated with NTA-SAM after incubating overnight in 0.1 mM NTA-SAM (Prochimia) solution in ethanol. Then, the tips were rinsed with plenty of ethanol, dried under nitrogen and incubated for 1 h in 40 mM NiSO₄ in a PBS solution and stored at 0-5° C.

AFM based on force/distance (FD)—A Resolve® AFM (Bruker) was used in “PeakForceTapping” mode. Rectangular cantilevers with nominal spring constants of approximately 0.06-0.12 N·m⁻¹ and a resonance frequency of approximately 18 kHz in water were chosen. All the AFM experiments were conducted in an imaging buffer solution at ambient temperature (approximately 24° C.). The adhesion charts were obtained by oscillating the functionalized tip at 0.25 kHz, with an amplitude of 25 nm, and by applying an imaging force of 100 pN. Topographies of 128×128 or 256×256 pixels were performed by digitizing 0.125 line per sec. The retraction speed was 1500 nm/sec and the contact time between the tip and the sample was 500 ms.

### Data Analysis

The force/distance (FD) curves from each interaction recognition experiment were saved and exported in text file format. NanoScope Analysis v1.9 and BiomecaAnalysis were used to convert the force/time curves into FD curves showing specific adhesion events. The force/distance curves obtained were then analyzed based on the Worm-Like Chain (WLC) model. This model is the most suitable and the most frequently used to describe the extension of polypeptides. The extension z of the macromolecule is linked with the retraction force F_{adh} by the equation:

$${F}_{adh} ( z ) = - \frac{{K}_{B} T}{{I}_{p}} ( \frac{z}{{I}_{c}} + 4 {( 1 - \frac{z}{{I}_{c}} )}^{- 2} - \frac{1}{4} )$$

wherein the persistence length lₚ is a direct measurement of the chain rigidity, l_{c} is the total contour length of the biomacromolecule and K_{B} is the Boltzmann constant.

The number of monomers in the polypeptide chains was then derived from the following equation:

$$N = \frac{{I}_{c}}{{I}_{p}}$$

### E.2/ Determination of the Orientation of the OprF Proteins in the Liposomal Membrane by AFM (Atomic Force Microscopy)

The OprF proteoliposome samples were adsorbed on a mica surface and analyzed by AFM using a probe functionalized by a Tris-Ni⁺-NTA group binding the N-terminal polyhistidine tag of OprF. The AFM analysis was performed with and without Triton® X-100 detergent. Triton® 1x solution was used to solubilize the OprF proteins of the liposomal membrane, thus exposing all the polyhistidine tags located inside the liposome and enabling them to be bound by the functionalized probe. The topographic images, acquired with and without Triton® X-100, showed OprF proteoliposomes on the sample surface. In the absence of Triton®, very few specific adhesion phenomena between the functionalized probe and the N-terminal polyhistidine tag of OprF occurred on the surface of the proteoliposomes, as shown by the corresponding adhesion charts illustrating the adhesion forces between 80 and 150 pN. On the other hand, in the presence of Triton®, numerous specific adhesion events were detected. On average, the functionalized probe has bound the polyhistidine tag of one OprF out of 6 without Triton®, and of 5 OprF proteins out of 6 with Triton®, demonstrating that the N-terminal polyhistidine tag of OprF was primarily located inside the liposome.

### E.3/ Determination of the Topology of OprF Proteins in the Liposomal Membrane by Trypsin Digestion and AFM

The OprF proteoliposomes purified by ultracentrifugation in a sucrose gradient were subjected to a limited proteolysis experiment in order to determine the topology of OprF in the liposomal membrane. The sequence of the OprF protein contains 32 trypsin cleavage sites. Without OprF membrane protection, trypsin generates peptides of a mass ranging from 146 to 4649 Da (PeptideCutter program). The result of trypsin digestion of the OprF proteoliposomes, visualized by Western Blot using an anti-histidine antibody, demonstrated that OprF adopts at least two different membrane topologies in the liposomes: a first topology wherein OprF is entirely inserted in the membrane and therefore protected from proteolysis, as the signal corresponding to the polyhistidine tag of the complete OprF protein did not disappear over time; and a second topology wherein about only half of the 6xHis-OprF protein is integrated in the membrane, as a smaller fragment of protein located between the molecular weight 20 and 25 kDa was generated over time.

These first observations were then corroborated and refined by AFM. The analysis of the force/distance (FD) curves showing specific adhesion phenomena in Triton® 1 x solution indicated that OprF adopts two different transmembrane topologies in the liposomal membrane, corresponding to its closed and open channel conformations. Based on the WLC model, 64% of the specific adhesion phenomena corresponded to 8 transmembrane domains (closed channel conformation) and 36% of the specific adhesion phenomena corresponded to 16 transmembrane domains (open channel conformation).

### E.4/ Study of the Pore-Forming Activity of OprF in the Proteoliposomes Using Negative Staining Electron Microscopy and AFM

The negative staining electron microscopy and the AFM analysis of the OprF proteoliposomes made it possible to visualize the pore-forming activity of OprF in the liposomal membrane. In the electron microscopy images, a series of “holes” of an average size of 9.5±4 nm corresponding to pores were observed through the membranes of the liposomes wherein OprF was reconstituted. Such a perforation of the liposomal membrane was not observed in the images of the control liposomes, incubated with the cell lysate and the reaction mixture of the acellular system without DNA (negative control). Moreover, the topographic AFM images of the surface of the OprF proteoliposomes also revealed the presence of pores surrounded by OprF proteins and having a mean diameter of 10 nm. Pore formation was therefore attributed to the activity of the OprF protein in the liposomal membrane.

1. A monoclonal antibody against the OprF protein of Pseudomonas aeruginosa or functional fragment of said antibody, comprising:
a heavy chain variable region having the three complementarity determining regions (CDR) having the following amino acid sequences, or sequences having at least 80% identity with these sequences:
VH-CDR1: GYXaa₁FXaa₂Xaa₃Xaa₄G (SEQ ID NO: 1) wherein Xaa₁ is a threonine residue or a serine residue, Xaa₂ is a serine residue or an asparagine residue, Xaa₃ is an arginine residue, a serine residue or a threonine residue and Xaa₄ is a phenylalanine residue or a tyrosine residue,
VH-CDR2: INAXaa₅TGKXaa₆ (SEQ ID NO: 2) wherein Xaa₅ is a glutamic acid residue or an aspartic acid residue and Xaa₆ is an alanine residue or a serine residue,
VH-CDR3: VR,
and a light chain variable region having the three CDRs having the following amino acid sequences, or sequences having at least 80% identity with these sequences:
VL-CDR1: SSVXaa₇TXaa₈Xaa₉ (SEQ ID NO: 3) wherein Xaa₇ is a threonine residue, an asparagine residue, a serine residue, an alanine residue or an arginine residue, Xaa₈ is an asparagine residue, a glycine residue or a serine residue and Xaa₉ is a tyrosine residue or a phenylalanine residue,
VL-CDR2: Xaa₁₀TS wherein Xaa₁₀ is a glycine residue, an arginine residue or an alanine residue,
VL-CDR3: QQGXaa₁₁Xaa₁₂Xaa₁₃ (SEQ ID NO: 4) wherein Xaa₁₁ is a histidine residue or an asparagine residue, Xaa₁₂ is a serine residue or a threonine residue and Xaa₁₃ is a valine residue or an isoleucine residue.

2. The monoclonal antibody or functional fragment of said antibody according to claim 1, wherein Xaa₃ is an arginine residue and Xaa₆ is an alanine residue.

3. The monoclonal antibody or functional fragment of said antibody according to claim 1, wherein Xaa₁ is a threonine residue, Xaa₅ is a glutamic acid residue and Xaa₁₃ is a valine residue.

4. The monoclonal antibody or functional fragment of said antibody according to claim 1, wherein
the complementarity determining regions (CDR) of the heavy chain variable region have the following respective amino acid sequences, or sequences having at least 80% identity with these sequences:
VH-CDR1:
(SEQ ID NO: 5)
GYTFSRFG,
VH-CDR2:
(SEQ ID NO: 12)
INAETGKA,
VH-CDR3:
VR
and/or the complementarity determining regions (CDR) of the light chain variable region have the following respective amino acid sequences, or sequences having at least 80% identity with these sequences:
VL-CDR1:
(SEQ ID NO: 16)
SSVTTNY,
VL-CDR2:
GTS
VL-CDR3:
(SEQ ID NO: 24)
QQGHSV.

5. The monoclonal antibody or functional fragment of said antibody according to claim 1, wherein:
the complementarity determining regions (CDR) of the heavy chain variable region have the following respective amino acid sequences, or sequences having at least 80% identity with these sequences:
VH-CDR1:
(SEQ ID NO: 6)
GYSFSSYG
VH-CDR2:
(SEQ ID NO: 13)
INADTGKS,
VH-CDR3:
VR
and/or the complementarity determining regions (CDR) of the light chain variable region have the following respective amino acid sequences, or sequences having at least 80% identity with these sequences:
VL-CDR1:
(SEQ ID NO: 17)
SSVTTGY
or
(SEQ ID NO: 16)
SSVTTNY
VL-CDR2:
GTS
VL-CDR3:
(SEQ ID NO: 25)
QQGHTI
or
(SEQ ID NO: 26)
QQGNTI.

6. The monoclonal antibody or functional fragment of said antibody according to claim 1, wherein:
the complementarity determining regions (CDR) of the heavy chain variable region have the following respective amino acid sequences, or sequences having at least 80% identity with these sequences:
VH-CDR1:
(SEQ ID NO: 7)
GYSFSTYG
or
(SEQ ID NO: 8)
GYSFSRYG
VH-CDR2:
(SEQ ID NO: 13)
INADTGKS
or
(SEQ ID NO: 14)
INADTGKA
VH-CDR3:
VR
and/or the complementarity determining regions (CDR) of the light chain variable region have the following respective amino acid sequences, or sequences having at least 80% identity with these sequences:
VL-CDR1:
(SEQ ID NO: 18)
SSVNTNY
or
(SEQ ID NO: 17)
SSVTTGY
or
(SEQ ID NO: 16)
SSVTTNY,
VL-CDR2:
GTS
VL-CDR3:
(SEQ ID NO: 25)
QQGHTI
or
(SEQ ID NO: 26)
QQGNTI.

7. The monoclonal antibody or functional fragment of said antibody according to claim 1, consisting of a single-chain variable fragment (scFv).

8. The monoclonal antibody or functional fragment of said antibody according to claim 7, wherein the heavy chain variable part and the light chain variable part are bound by a binding peptide.

9. The monoclonal antibody or functional fragment of said antibody according to claim 8, comprising a pair of sequences selected from the group consisting of the following pairs of sequences, SEQ ID NO: 28 and SEQ ID NO: 29, SEQ ID NO: 30 and SEQ ID NO: 31, SEQ ID NO: 32 and SEQ ID NO: 33, SEQ ID NO: 34 and SEQ ID NO: 35, SEQ ID NO: 36 and SEQ ID NO: 37, SEQ ID NO: 38 and SEQ ID NO: 39, SEQ ID NO: 40 and SEQ ID NO: 41, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, and pairs of sequences having at least 80% identity with one of these pairs of sequences.

10. The monoclonal antibody or functional fragment of said antibody according to claim 1, consisting of a chimeric or humanized antibody or antibody fragment.

11. A nucleic acid molecule coding for a monoclonal antibody or functional fragment of said antibody according to claim 1.

12. An expression vector comprising a nucleic acid molecule according to claim 11.

13. A host cell comprising a nucleic acid molecule according to claim 11.

14. A method for preparing the monoclonal antibody or functional fragment of said antibody according to claim 1, the method comprising:
culturing host cells comprising a nucleic acid molecule coding for the monoclonal antibody or functional fragment of said antibody under conditions enabling the expression of said monoclonal antibody or fragment of said antibody; and
recovering said antibody or functional fragment of said antibody thus produced.

15. A method for preparing the monoclonal antibody or functional fragment of said antibody according to claim 1, comprising successive steps of:
producing proteoliposomes containing the OprF protein of Pseudomonas aeruginosa,
inoculating a non-human mammal with said proteoliposomes,
constructing a bank of antibodies or antibody fragments from RNA extracted from cells of said mammal,
screening said bank with respect to said proteoliposomes, by an expression technique,
and selecting the clones which are reactive with respect to said proteoliposomes.

16. A pharmaceutical composition for combatting bacterial infections, comprising the monoclonal antibody or functional fragment of said antibody according to claim 1 as an active substance, in a pharmaceutically acceptable vehicle.

17. A method of preventively or curatively treating an infection in an individual in need thereof, comprising administering to said individual a therapeutically effective dose of the monoclonal antibody or functional fragment of said antibody according to claim 1.

18. The method of claim 17, wherein the infection is a bacterial Pseudomonas aeruginosa infections.

19. The method of claim 18, wherein the infection is a lung infection.

20. A method for in vitro or ex vivo detection of the bacterium Pseudomonas aeruginosa in a body fluid from an individual, the method comprising binding the monoclonal antibody or functional fragment of said antibody according to claim 1 to the bacterium.

21. A kit for the in vitro or ex vivo detection of the bacterium Pseudomonas aeruginosa in a body fluid from an individual, comprising the monoclonal antibody or functional fragment of said antibody according to claim 1, reagents, and instructions for implementing a method for detecting, in vitro or ex vivo, the bacterium Pseudomonas aeruginosa in a body fluid from an individual, by means of said monoclonal antibody or functional fragment of said antibody.


SEQUENCE LISTING (79 sequences)

SEQ ID NO: 1 - protein, 8 aa, Macaca fascicularis
  MISC_FEATURE (3)..(3): Xaa is threonine or serine
  MISC_FEATURE (5)..(5): Xaa is serine or asparagine
  MISC_FEATURE (6)..(6): Xaa is arginine or serine or threonine
  MISC_FEATURE (7)..(7): Xaa is phenylalanine or tyrosine
GYXFXXXG

SEQ ID NO: 2 - protein, 8 aa, Macaca fascicularis
  MISC_FEATURE (4)..(4): Xaa is glutamic acid or aspartic acid
  MISC_FEATURE (8)..(8): Xaa is alanine or serine
INAXTGKX

SEQ ID NO: 3 - protein, 7 aa, Macaca fascicularis
  MISC_FEATURE (4)..(4): Xaa is threonine or asparagine or serine or alanine or arginine
  MISC_FEATURE (6)..(6): Xaa is asparagine or glycine or serine
  MISC_FEATURE (7)..(7): Xaa is tyrosine or phenylalanine
SSVXTXX

SEQ ID NO: 4 - protein, 6 aa, Macaca fascicularis
  MISC_FEATURE (4)..(4): Xaa is histidine or asparagine
  MISC_FEATURE (5)..(5): Xaa is serine or threonine
  MISC_FEATURE (6)..(6): Xaa is valine or isoleucine
QQGXXX

SEQ ID NO: 5 - protein, 8 aa, Macaca fascicularis
GYTFSRFG

SEQ ID NO: 6 - protein, 8 aa, Macaca fascicularis
GYSFSSYG

SEQ ID NO: 7 - protein, 8 aa, Macaca fascicularis
GYSFSSYG

SEQ ID NO: 8 - protein, 8 aa, Macaca fascicularis
GYSFSRYG

SEQ ID NO: 9 - protein, 8 aa, Macaca fascicularis
GYSFNTYG

SEQ ID NO: 10 - protein, 8 aa, Macaca fascicularis
GYSFSTFG

SEQ ID NO: 11 - protein, 15 aa, Artificial sequence
  Peptide linker between variable region of heavy chain and variable region of light chain of the antibodies
GGGGSGGGGSGGGGS

SEQ ID NO: 12 - protein, 8 aa, Macaca fascicularis
INAETGKA

SEQ ID NO: 13 - protein, 8 aa, Macaca fascicularis
INADTGKS

SEQ ID NO: 14 - protein, 8 aa, Macaca fascicularis
INADTGKA

SEQ ID NO: 15 - protein, 8 aa, Macaca fascicularis
INAETGKS

SEQ ID NO: 16 - protein, 7 aa, Macaca fascicularis
SSVTTNY

SEQ ID NO: 17 - protein, 7 aa, Macaca fascicularis
SSVTTGY

SEQ ID NO: 18 - protein, 7 aa, Macaca fascicularis
SSVNTNY

SEQ ID NO: 19 - protein, 7 aa, Macaca fascicularis
SSVSTNY

SEQ ID NO: 20 - protein, 7 aa, Macaca fascicularis
SSVATGF

SEQ ID NO: 21 - protein, 7 aa, Macaca fascicularis
SSVSTSY

SEQ ID NO: 22 - protein, 7 aa, Macaca fascicularis
SSVRTGY

SEQ ID NO: 23 - protein, 7 aa, Macaca fascicularis
SSVSTGY

SEQ ID NO: 24 - protein, 6 aa, Macaca fascicularis
QQGHSV

SEQ ID NO: 25 - protein, 6 aa, Macaca fascicularis
QQGHTI

SEQ ID NO: 26 - protein, 6 aa, Macaca fascicularis
QQGNTI

SEQ ID NO: 27 - protein, 6 aa, Macaca fascicularis
QQGHSI

SEQ ID NO: 28 - protein, 120 aa, Macaca fascicularis
QVQLEQSGAEVKQPGASVKVSCKASGYTFSTFGMNWVRQAPGQRPEWMGWINADTGKSTYAPGFKERFTFSLDTSVSTAYLTISSLKAEDTAVYYCVRSCRGGVCQLYHWGQGGLITVSS

SEQ ID NO: 29 - protein, 108 aa, Macaca fascicularis
DIELTQSPTSIAVSQGERVTISCTATSSVATGFLHWYQQKPGFPPRLLVYGTSSLPSGVPARFSGSGSGTSYTLTISSMEPEDAAHYYCQQGHSIPLTFGGGTKVEIK

SEQ ID NO: 30 - protein, 120 aa, Macaca fascicularis; identical to SEQ ID NO: 28

SEQ ID NO: 31 - protein, 108 aa, Macaca fascicularis; identical to SEQ ID NO: 29

SEQ ID NO: 32 - protein, 120 aa, Macaca fascicularis
QVQLEQSGAEVKHPGASVKVSCKASGYSFSTFGMNWVRQAPGQRPEWMGWINADTGKSTYAPGFKERFTFSLDTSVSTAYLTISSLKAEDTAVYYCVRSCRGGVCQLYHWGQGVLITVSS

SEQ ID NO: 33 - protein, 108 aa, Macaca fascicularis
EIELTQSPTSMAVSQGERVTISCTASSSVNTNYLHWYQQKPGFPPRLLVYGTSSLVAGVPARFSGSGSGTSYTLTISSMEAEDAAYYYCQQGNTIPLTFGGGTKVEIK

SEQ ID NO: 34 - protein, 120 aa, Macaca fascicularis
EVQLVQSGAEVKQPGASVKVSCKASGYSFSTYGMNWVRQAPGQRPEWMGWINADTGKSTYAPGFKERFTFSLDTSVSTAYLTISSLKAEDTAVYYCVRSCRGGVCQLYHWGQGVLITVSS

SEQ ID NO: 35 - protein, 108 aa, Macaca fascicularis
DIELTQSPTSMAVSQGERVTISCTASSSVSTGYFHWYQQKPGFPPRLLVYATSSLPSGVPARFSGSGSGTSYTLTISNVEAEDAANYYCQQGHSIPLTFGGGTTVEIK

SEQ ID NO: 36 - protein, 120 aa, Macaca fascicularis
QVQLEQSGAEVKQPGASVKVSCKASGYSFNTYGMNWVRQVPGQRPEWMGWINAETGKSTYAPGFKERFTFSLDTSISTAYLKISSLKAEDTAVYYCVRSCRGGVCQLYHWGQGVLITVSS

SEQ ID NO: 37 - protein, 120 aa, Macaca fascicularis; identical to SEQ ID NO: 36

SEQ ID NO: 38 - protein, 120 aa, Macaca fascicularis
QVQLEQSGAEVKQPGASVKVSCKASGYSFSSYGMNWVRQAPGQRPEWMGWINADTGKSTYAPGFKERFTFSLDTSVSTAYLTISSLKAEDTAVYYCVRSCRGGVCQLYHWGQGVLITVSS

SEQ ID NO: 39 - protein, 108 aa, Macaca fascicularis
DIELTQSPTSIAVSQGERVTISCTASSSVTTGYFHWYQQKPGFPPRLLVYGTSSLPSGVPARFSGSGSGTSYTLTISNMEAEDAAHYYCQQGHTIPLTFGGGTKVEIK

SEQ ID NO: 40 - protein, 120 aa, Macaca fascicularis
EVQLVQSGAEVKQPGASVKVSCKASGYSFNTYGMNWVRQVPGQRPEWMGWINAETGKSTYAQDFKERFTFTMDTSISTAYLQISSLKAEDTAVYYCVRSCRGGVCQLYYWGQGALVTVSS

SEQ ID NO: 41 - protein, 108 aa, Macaca fascicularis
DIELTQSPTSVAVSQGERVTISCTASSSVSTSYLHWYQQKPGFPPRLLVYATSSLPSGVPARFSGSGSGTSYTLTISNMEAEDAANYYCQQGNTIPLTFGGGTKVEIK

SEQ ID NO: 42 - protein, 120 aa, Macaca fascicularis
QVQLEQSGAEVKQPGASVKVSCKASGYSFSSYGMNWVRQAPGQRPEWLGWINADTGKSTYAPGFKERFTFSLDTSVSTAYLTISSLKAEDTAVYYCVRSCRGGVCQLYHWGQGVLITVSS

SEQ ID NO: 43 - protein, 108 aa, Macaca fascicularis
EIELTQSPTSIAVSRGERVTVSCMASSSVRTGYLHWYQQKPGFPPRLLVYGTSSLPSGVPARFSGSGSGTSYTLTISSVEPEDVAHYYCQQGHTIPLTFGGGTKVEIK

SEQ ID NO: 44 - protein, 120 aa, Macaca fascicularis
QVQLEQSGGGLIQPGGSLKVSCAASGYTFSRFGMSWVRQAPGQRPEWMGWINAETGKATYAQGFKQRFTFSMDTSISTAYLQISSLKAEDTAVYYCVRSCRGGVCQLYHWGQGALVTVSS

SEQ ID NO: 45 - protein, 108 aa, Macaca fascicularis
DIELTQSPTSITVSQGERVTISCTASSSVTTNYLHWYQQKPGFPPRLLVYGTSSLPSEVPARFSGSGSGTSYMLTISTMEAEDAAIYYCQQGHSVPLTFGGGTKVEIK

SEQ ID NO: 46 - protein, 120 aa, Macaca fascicularis
QVQLEQSGAEVKQPGASVKVSCKASGYSFNTYGMNWVRQAPGQRPEWMGWINADTGKSTYAPGFKERFTFSLDTSVSTAYLTISSLKAEDTAVYFCVRSCRGGVCQLYHWGQGVLVTVSS

SEQ ID NO: 47 - protein, 108 aa, Macaca fascicularis
DIELTQSPTSMAVSQGERVTISCTASSSVSTNYLHWYQQKPGFPPRLLVYRTSSLAPGVPARFSGSGSGTSYTLTISNMEAEDAAHYYCQQGNTIPLTFGGGTKVEIK

SEQ ID NO: 48 - protein, 120 aa, Macaca fascicularis
QVQLEQSGAEVKHPGASVKVSCKASGYSFSTYGMNWVRQAPGQRPEWMGWINADTGKSTYAPGFKERFTFSLDTSVSTAYLTISSLKAEDTAVYYCVRSCRGGVCQLYHWGQGVLITVSS

SEQ ID NO: 49 - protein, 108 aa, Macaca fascicularis
ELQMTQSPTSMAVSQGERVTISCTASSSVNTNYLHWYQQKPGFPPRLLVYGTSSLVAGVPARFSGSGSGTSYTLTISNMEAEDAANYYCQQGNTIPLTFGGGTKVEIK

SEQ ID NO: 50 - protein, 243 aa, Macaca fascicularis
QVQLEQSGAEVKQPGASVKVSCKASGYTFSTFGMNWVRQAPGQRPEWMGWINADTGKSTYAPGFKERFTFSLDTSVSTAYLTISSLKAEDTAVYYCVRSCRGGVCQLYHWGQGGLITVSSGGGGSGGGGSGGGGSDIELTQSPTSIAVSQGERVTISCTATSSVATGFLHWYQQKPGFPPRLLVYGTSSLPSGVPARFSGSGSGTSYTLTISSMEPEDAAHYYCQQGHSIPLTFGGGTKVEIK

SEQ ID NO: 51 - protein, 243 aa, Macaca fascicularis; identical to SEQ ID NO: 50

SEQ ID NO: 52 - protein, 243 aa, Macaca fascicularis
QVQLEQSGAEVKHPGASVKVSCKASGYSFSTFGMNWVRQAPGQRPEWMGWINADTGKSTYAPGFKERFTFSLDTSVSTAYLTISSLKAEDTAVYYCVRSCRGGVCQLYHWGQGVLITVSSGGGGSGGGGSGGGGSEIELTQSPTSMAVSQGERVTISCTASSSVNTNYLHWYQQKPGFPPRLLVYGTSSLVAGVPARFSGSGSGTSYTLTISSMEAEDAAYYYCQQGNTIPLTFGGGTKVEIK

SEQ ID NO: 53 - protein, 243 aa, Macaca fascicularis; identical to SEQ ID NO: 52

SEQ ID NO: 54 - protein, 243 aa, Macaca fascicularis
QVQLEQSGAEVKQPGASVKVSCKASGYSFNTYGMNWVRQVPGQRPEWMGWINAETGKSTYAPGFKERFTFSLDTSISTAYLKISSLKAEDTAVYYCVRSCRGGVCQLYHWGQGVLITVSSGGGGSGGGGSGGGGSDIELTQSPTSMAVSQGERVTISCTASSSVNTNYLHWYQQKPGFPPRLLVYGTSSLPSGVPARFSGSGSGTSYTLTISSMEAEDAAHYYCQQGNTIPLTFGGGTKVEIK

SEQ ID NO: 55 - protein, 243 aa, Macaca fascicularis
QVQLEQSGAEVKQPGASVKVSCKASGYSFSSYGMNWVRQAPGQRPEWMGWINADTGKSTYAPGFKERFTFSLDTSVSTAYLTISSLKAEDTAVYYCVRSCRGGVCQLYHWGQGVLITVSSGGGGSGGGGSGGGGSDIELTQSPTSIAVSQGERVTISCTASSSVTTGYFHWYQQKPGFPPRLLVYGTSSLPSGVPARFSGSGSGTSYTLTISNMEAEDAAHYYCQQGHTIPLTFGGGTKVEIK

SEQ ID NO: 56 - protein, 243 aa, Macaca fascicularis
EVQLVQSGAEVKQPGASVKVSCKASGYSFNTYGMNWVRQVPGQRPEWMGWINAETGKSTYAQDFKERFTFTMDTSISTAYLQISSLKAEDTAVYYCVRSCRGGVCQLYYWGQGALVTVSSGGGGSGGGGSGGGGSDIELTQSPTSVAVSQGERVTISCTASSSVSTSYLHWYQQKPGFPPRLLVYATSSLPSGVPARFSGSGSGTSYTLTISNMEAEDAANYYCQQGNTIPLTFGGGTKVEIK

SEQ ID NO: 57 - protein, 243 aa, Macaca fascicularis
QVQLEQSGAEVKQPGASVKVSCKASGYSFSSYGMNWVRQAPGQRPEWLGWINADTGKSTYAPGFKERFTFSLDTSVSTAYLTISSLKAEDTAVYYCVRSCRGGVCQLYHWGQGVLITVSSGGGGSGGGGSGGGGSEIELTQSPTSIAVSRGERVTVSCMASSSVRTGYLHWYQQKPGFPPRLLVYGTSSLPSGVPARFSGSGSGTSYTLTISSVEPEDVAHYYCQQGHTIPLTFGGGTKVEIK

SEQ ID NO: 58 - protein, 243 aa, Macaca fascicularis; identical to SEQ ID NO: 57

SEQ ID NO: 59 - protein, 243 aa, Macaca fascicularis
QVQLEQSGAEVKQPGASVKVSCKASGYSFNTYGMNWVRQAPGQRPEWMGWINADTGKSTYAPGFKERFTFSLDTSVSTAYLTISSLKAEDTAVYFCVRSCRGGVCQLYHWGQGVLVTVSSGGGGSGGGGSGGGGSDIELTQSPTSMAVSQGERVTISCTASSSVSTNYLHWYQQKPGFPPRLLVYRTSSLAPGVPARFSGSGSGTSYTLTISNMEAEDAAHYYCQQGNTIPLTFGGGTKVEIK

SEQ ID NO: 60 - protein, 243 aa, Macaca fascicularis
QVQLEQSGAEVKHPGASVKVSCKASGYSFSTYGMNWVRQAPGQRPEWMGWINADTGKSTYAPGFKERFTFSLDTSVSTAYLTISSLKAEDTAVYYCVRSCRGGVCQLYHWGQGVLITVSSGGGGSGGGGSGGGGSELQMTQSPTSMAVSQGERVTISCTASSSVNTNYLHWYQQKPGFPPRLLVYGTSSLVAGVPARFSGSGSGTSYTLTISNMEAEDAANYYCQQGNTIPLTFGGGTKVEIK

SEQ ID NO: 61 - protein, 330 aa, Homo sapiens [sequence omitted]

SEQ ID NO: 62 - DNA, 1141 bp, Macaca fascicularis
aggtatcacgaggccctttcgtcttcacctcgagagcgggcagtgagcgcaacgcaattaatgtgagttagctcactcattaggcaccccaggctttacactttatgctcccggctcgtatgttgtgtggaattgtgagcggataacaatttcacacagaattcattaaagaggagaaattaaccatgaaatacctattgcctacggcagccgctggcttgctgctgctggcggcccagccggccatggcccaggtacagctcgagcagtcaggggctgaggtgaagcagccgggggcctcagtgaaggtctcctgcaaggcttctggatacagcttcagtacgtatggtatgaattgggtgcggcaagcccctggacaacggcctgagtggatggggtggatcaacgccgacactgggaagtcaacatatgccccgggcttcaaagaacgatttaccttctccttggacacctctgtcagcacggcatatctgacgatcagcagcctaaaggctgaggacacggccgtctattactgtgtgagatcgtgtcgtggtggtgtctgccagctttaccactggggccagggagtcctgatcaccgtctcgtcaggcggcggcggcagcggcggcggcggcagcggcggcggcggcagcgaaatcgagctcactcagtctccaacatccatagctgtctcccaaggagaaagagtcaccatctcctgcacggccagctcaagtgtaagcaccagctacttccactggtaccaacagaagccaggattccctccaaggcttcttgtctatgcaacatcaagtctgccttctggagtcccagccaggttcagtggcagcggttctgggacctcttacacgctcacaatcagcagcatggaggctgaagatgctgcaaactattactgtcaacaggggcatagcattcccctcactttcggcggagggaccaaagtggagatcaaacgagctgtggctccaccatctgtcgcggccgcaggttctgaacaaaagctgatctcagaagaagatctatcccatcatcaccatcatcattagggatccaaagatatcagagctgaaactgttgaaagttgtttagcaaaatcccatcagtcggcgcgaag

SEQ ID NO: 63 - DNA, 1183 bp, Macaca fascicularis
gggggtgtgcggcagcggccatggcccaggtacagctcgagcagtcaggggctgaggtgaagcacccgggggcctcagtgaaggtctcctgcaaggcttctggatacagcttcagtacctttggtatgaattgggtgcggcaagcccctggacaacggcctgagtggatgggatggatcaacgccgacactgggaagtcaacatatgccccgggcttcaaagaacgatttaccttctccttggacacctctgtcagcacggcatatctgacgatcagcagcctaaaggctgaggacacggccgtctattactgtgtgagatcgtgtcgtggtggtgtctgccagctttaccactggggccagggagtcctgatcaccgtctcgtcaggcggcggcggcagcggcggcggcggcagcggcggcggcggcagcgacatcgagctcactcagtctccagcatccatagctgtctcccagggagaaagagtcaccatctcctgcacggccacctcaagtgtagccaccggcttcttgcactggtaccaacagaagccaggattccctccaaggcttcttgtctatggaacgtcaagtctgccttctggagtcccagccaggttcagtggcagcggttctgggacctcttacacgctcacaatcagcagcatggagcctgaagatgctgcacactattactgtcagcagggacatagcattcccctcactttcggcggagggaccaaagtggagatcaaacgagctgtggctccaccatctgtcgcggccgcaggttctgaacaaaagctgatctcagaagaagatctatcccatcatcaccatcatcattagggatccaaagatatcagagctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgaaggtggcggttctgaaggtggcggttctgaaggtggcggtactaaacctcctgagtacggtgatacacctattccgggctatacttatatcaacccttt

SEQ ID NO: 64 - DNA, 960 bp, Macaca fascicularis
gccggcgaattaaccatgaaatacctattgcccaggtacagctcgagcagtcaggggctgaggtgaagcacccgggggcctcagtgaaggtctcctgcaaggcttctggatacagcttcagtacctttggtatgaattgggtgcggcaagcccctggacaacggcctgagtggatgggatggatcaacgccgacactgggaagtcaacatatgccccgggcttcaaagaacgatttaccttctccttggacacctctgtcagcacggcatatctgacgatcagcagcctaaaggctgaggacacggccgtctattactgtgtgagatcgtgtcgtggtggtgtctgccagctttaccactggggccagggagtcctgatcaccgtctcgtcaggcggcggcggcagcggcggcggcggcagcggcggcggcggcagcgaaatcgagctcactcagtctccaacatccatggctgtctcccaaggagaaagagtcaccatctcctgcacggccagctcaagtgtaaacaccaactacttgcactggtaccaacagaagccaggatttcctccaaggcttcttgtctatggaacatcaagtctggtggctggagtcccagccaggttcagtggcagcggttctgggacctcttacactctcacaatcagcagcatggaggctgaggatgctgcatactattactgtcagcaggggaataccattccactcactttcggcggagggaccaaggtggagatcaaacgagctgtggctgcaccatctgtcgcggccgcaggttctgaacaaaagctgatctcagaagaagatctatcccatcatcaccatcatcattagggatccaaagatatcagagctgaactgttgaaagttgtttagcaaaatcccatacagaaaattcatttactaacgtctggaagcgacaaaaaaaaatcttttagaa

SEQ ID NO: 65 - DNA, 946 bp, Macaca fascicularis
tgtcatggaaatacctattgccgaggtgcagctggtgcagtctggggctgaggtgaagcagccgggggcctcagtgaaggtctcctgcaaggcttctggatacagcttcagtacgtatggtatgaattgggtgcggcaagcccctggacaacggcctgagtggatggggtggatcaacgccgacactgggaagtcaacatatgccccgggcttcaaagaacgatttaccttctccttggacacctctgtcagcacggcatatctgacgatcagcagcctaaaggctgaggacacggccgtctattactgtgtgagatcgtgtcgtggtggtgtctgccagctttaccactggggccagggagtcctgatcaccgtctcgtcaggcggcggcggcagcggcggcggcggcagcggcggcggcggcagcgatatcgagctcacgcagtctccaacatccatggctgtctcccaaggagaaagagtcaccatctcctgcacggccagctcaagtgtaagcaccggctacttccactggtaccaacagaagccaggattccctccaaggcttcttgtctatgcaacatcaagtctgccttctggagtcccagccaggttcagtggcagcggttccgggacctcttacacgcttacaatcagcaacgtggaggctgaagatgctgcaaactattactgtcaacaggggcatagcattcccctcactttcggcggagggaccacagtggagatcaaacgagccgtggctgcaccatctgtcgcggccgcagttctgaacaaaagctgatctcagaagaagatctatcccatcatcaccatcatcattagggatccaaagatatcagagctgaactgttgaaagttgtttagcaaaatcccatacagaaaattcatttactaacgtctggaagcgcaaaaaaaacttttaaaa

SEQ ID NO: 66 - DNA, 950 bp, Macaca fascicularis
tcggccaattaaccatgaaatacctattgcccaggtacagctcgagcagtcaggggctgaggtgaagcagccgggggcctcagtgaaggtctcctgcaaggcttctggatacagtttcaatacctatggtatgaattgggtgcgacaagtccctggacaacggcctgagtggatgggatggatcaacgccgaaactgggaagtcaacatatgccccgggcttcaaggaacgatttaccttctccttggacacctctatcagtacggcatatctgaagattagtagcctaaaggctgaggacacggccgtctattattgtgtgagatcgtgtagaggtggtgtctgccagctttaccactggggccagggagtcctgatcaccgtctcctcaggcggcggcggcagcggcggcggcggcagcggcggcggcggcagcgacatcgagctcactcagtctccaacatccatggctgtctcccaaggagaaagagtcaccatctcctgcacggccagctcaagtgtaaacaccaactacttgcactggtaccaacagaagccaggattccctccaaggcttcttgtctatggaacatcaagtctgccttctggagtcccagccaggttcagtggcagcggttctgggacctcttacacgctcacaatcagcagcatggaggctgaagatgctgcacactattactgtcagcaggggaataccattccactcactttcggcggagggaccaaagtggagatcaaacgagctgtggctgcaccatctgtcgcggccgcagttctgaacaaaagctgatctcagaagaagatctatcccatcatcaccatcatcattagggatcaaagatatcagagctgaaactgtgaaagtgtttagcaaatcccatacagaaatcatttactaacgtctgaagagacaaaaaaaactttagaaa

SEQ ID NO: 67 - DNA, 954 bp, Macaca fascicularis
ggggcgaattaaccatgaaatacctattgcccaggtacagctcgagcagtcaggggctgaggtgaagcagccgggggcctcagtgaaggtctcctgcaaggcttctggatacagcttcagttcctatggtatgaattgggtgcggcaagcccctggacaacggcctgagtggatgggatggatcaacgccgacactgggaagtcaacatatgccccgggcttcaaagaacgatttaccttctccttggacacctctgtcagcacggcatatctgacgatcagcagcctaaaggctgaggacacggccgtctattactgtgtgagatcgtgtcgtggtggtgtctgccagctttaccactggggccagggagtcctgatcaccgtctcgtcaggcggcggcggcagcggcggcggcggcagcggcggcggcggcagcgatatcgagctcacacagtctccaacatccatagctgtctcccaaggagaaagagtcaccatctcctgcacggccagctcaagtgtaaccaccggctacttccactggtaccaacagaagccaggattccctccaaggcttcttgtctatggaacctcaagtctgccttctggagtccccgccaggttcagtggcagcggttctgggacctcttacacgctcacaatcagcaacatggaggctgaagatgctgcacactattactgtcagcaggggcataccattcccctcactttcggcggagggaccaaagtggagatcaaacgagctgtggctgcaccatctgtcgcggccgcagttctgaacaaaagctgatctcagaagaagatctatcccatcatcaccatcatcattagggatccaaagatatcagagctgaactgttgaagttgtttagcaaatcccatacagaaaattcatttactaacgtctggaagcgcaaaaaaaatctttaagaa

SEQ ID NO: 68 - DNA, 946 bp, Macaca fascicularis
taaccatgaaatacctattgccgaggtgcagctggtgcagtctggggctgaggtgaagcagccgggggcctcagtgaaggtctcctgcaaggcttctggatacagtttcaatacctatggtatgaattgggtgcgacaagtccctggacaacggcctgagtggatgggatggatcaacgccgaaactgggaagtcaacatatgcccaggacttcaaggaacgatttaccttcaccatggacacgtctatcagtacggcgtatctgcagattagtagcctaaaggctgaggacacggccgtctattattgtgtgagatcgtgtagaggtggtgtctgccaactttattactggggccagggagccctggtcaccgtctcctcaggcggcggcggcagcggcggcggcggcagcggcggcggcggcagcgacatcgagctcacgcagtctccaacatccgtggctgtctcccaaggagagagagtcaccatctcctgcacggccagttcaagtgtaagcaccagctacttgcactggtaccaacagaagccaggattccctccaaggcttcttgtctatgcaacatcaagtctgccttctggagtcccagccaggttcagtggcagcggttctgggacctcttacacactcacaattagcaacatggaggctgaagatgctgcaaactattactgtcagcaggggaataccattccactcactttcggcggagggaccaaggtggagatcaaacgagctgtggctgcaccatctgtcgcggccgcagttctgaacaaaagctgatctcagaagaagatctatcccatcatcaccatcatcattagggatccaaagatatcagagctgaactgttgaaagttgtttagcaaaatcccatacagaaaattcatttactaacgtctggaagaaaccaaaaaaaactttagg

SEQ ID NO: 69 - DNA, 950 bp, Macaca fascicularis
tagcatgaaatacctattgcccaggtacagctcgagcagtcaggggctgaggtgaagcagccgggggcctcagtgaaggtctcctgcaaggcttctggatacagcttcagttcctatggtatgaattgggtgcggcaagcccctggacaacggcctgagtggctgggatggatcaacgccgacactgggaagtcaacatatgccccgggcttcaaagaacgatttaccttctccttggacacctctgtcagcacggcatatctgacgatcagcagcctaaaggctgaggacacggccgtctattactgtgtgagatcgtgtcgtggtggtgtctgccagctttaccactggggccagggagtcctgatcaccgtctcgtcaggcggcggcggcagcggcggcggcggcagcggcggcggcggcagcgaaatcgagctcacgcagtctccaacatccatagctgtctcccggggagaaagagtcaccgtctcctgcatggccagctcaagtgtgaggaccggctacttgcattggtaccaacagaagccaggattccccccaaggcttctagtctatggaacatcaagtctgccttctggagtcccagccaggttcagtggcagcggttctgggacctcttacacgctcaccatcagcagcgtggagcctgaagatgttgcacactattactgtcaacaggggcataccattcccctcactttcggcggagggaccaaagtggagatcaaacgagccgtggctgcaccatctgtcgcggccgcaggttctgaacaaaagctgatctcagaagaagatctatcccatcatcaccatcatcattagggatccaaagatatcagagctgaactgttgaaagttgtttagcaaaatcccatacagaaaattcatttactaacgtctggaagacgcacaaaaaaaactttagagaa

SEQ ID NO: 70 - DNA, 948 bp, Macaca fascicularis
acttaaccatgaaatacctattgcccaggtacagctcgagcagtcagggggaggcttgatccagcctggagggtccctgaaggtctcctgtgcagcctctggatacaccttcagtaggtttggcatgagttgggtgcgacaagcccctggacaacggcctgagtggatggggtggatcaacgccgaaactgggaaggcaacatatgcccagggcttcaaacaacgatttaccttctccatggacacctctatcagtacggcgtatctgcagattagcagcctaaaggctgaggacacggccgtctattattgtgtgagatcgtgtagaggtggtgtctgccaactttatcactggggccagggagccctggtcaccgtctcctcaggcggcggcggcagcggcggcggcggcagcggcggcggcggcagcgacatcgagctcacccagtctccaacatccataactgtctcccaaggagaaagagtcaccatctcctgcacggccagctcaagtgtaaccaccaactacttgcactggtaccaacagaagccaggattccctccaaggcttcttgtctatggaacatcaagtctgccttctgaagtcccagccaggttcagcggcagcggttctgggacctcttacatgctcacaatcagcactatggaggctgaagatgctgcaatctattactgtcagcaggggcatagcgttcccctcactttcgggggggggaccaaagtggagatcaaacgagctgtggctccaccatctgtcgcggccgcagttctgaacaaaagctgatctcagaagaagatctatcccatcatcaccatcatcattagggatccaagatatcagagctgaactgttgaaagttgtttagcaaatcccatacagaaaattcatttactaacgtctggaagagcaaaaaaacacttttgaaa

SEQ ID NO: 71 - DNA, 948 bp, Macaca fascicularis
atccatgaaatacctattgcccaggtacagctcgagcagtcaggggctgaggtgaagcagccgggggcctcagtgaaggtctcctgcaaggcttctggatacagcttcaatacctatggtatgaattgggtgcggcaagcccctggacaacggcctgagtggatgggatggatcaacgccgacactgggaagtcaacatatgccccgggcttcaaagaacgatttaccttctccttggacacctctgtcagcacggcatatctgacgatcagcagcctaaaggctgaggacacggccgtctatttctgtgtgagatcgtgtcgtggtggtgtctgccagctttaccactggggccagggagtcctggtcaccgtctcctcaggcggcggcggcagcggcggcggcggcagcggcggcggcggcagcgacatcgagctcacgcagtctccaacatccatggctgtctcccaaggagaaagagtcaccatctcctgtacggccagctcaagtgtaagcaccaactacttgcactggtaccaacagaagccaggatttcctccaaggcttcttgtctatagaacatcaagtctggctcctggagtcccagccaggttcagtggcagcggttctgggacctcttacacactcacaattagcaacatggaggctgaagatgctgcacactattactgtcagcaggggaataccattccactcactttcggcggagggaccaaagtggagatcaaacgagctgtggctccaccatctgtcgcggccgcagttctgaacaaaagctgatctcagaagaagatctatcccatcatcaccatcatcattagggatccaaagatatcagagctgaaactgttgaaagttgtttagcaaaatcccatacagaaaattcatttactaacgtctggaagacgcaaaaaaaaactttagaaa

SEQ ID NO: 72 - DNA, 958 bp, Macaca fascicularis
aacgccaattaccatgaaatacctattgcccaggtacagctcgagcagtcaggggctgaggtgaagcacccgggggcctcagtgaaggtctcctgcaaggcttctggatacagcttcagtacctatggtatgaattgggtgcggcaagcccctggacaacggcctgagtggatgggatggatcaacgccgacactgggaagtcaacatatgccccgggcttcaaagaacgatttaccttctccttggacacctctgtcagcacggcatatctgacgatcagcagcctaaaggctgaggacacggccgtctattactgtgtgagatcgtgtcgtggtggtgtctgccagctttaccactggggccagggagtcctgatcaccgtctcgtcaggcggcggcggcagcggcggcggcggcagcggcggcggcggcagcgagctccagatgacccagtctccaacatccatggctgtctcccaaggagaaagagtcaccatctcctgcacggccagctcaagtgtaaacaccaactacttgcactggtaccaacagaagccaggatttcctccaaggcttcttgtctatggaacatcaagtctggtggctggagtcccagccaggttcagtggcagcggttctgggacctcctacacactcacaattagcaacatggaggctgaagatgctgcaaactattactgtcagcaggggaataccattccactcactttcggcggagggaccaaggtggagatcaaacgagctgtggctgcaccatctgtcgcggccgcagttctgaacaaaagctgatctcagaagaagatctatcccatcatcaccatcatcattagggatccaaagatatcagagctgaactgttgaaagttgtttagcaaaatcccatacagaaaattcatttactaacgtctggaaagaaacaacaaaaacactttaagaa

SEQ ID NO: 73 - protein, 243 aa, Macaca fascicularis
QVQLEQSGAEVKQPGASVKVSCKASGYSFSTYGMNWVRQAPGQRPEWMGWINADTGKSTYAPGFKERFTFSLDTSVSTAYLTISSLKAEDTAVYYCVRSCRGGVCQLYHWGQGVLITVSSGGGGSGGGGSGGGGSDIELTQSPTSIAVSQGERVTISCTASSSVNTSYLHWYQQKPGFPPRLLVYGTSSLPSGVPARFSGSGSGTSYTLTISSMEPEDAAHYYCQQGHTIPLTFGGGTKVEIK

SEQ ID NO: 74 - protein, 108 aa, Macaca fascicularis
DIELTQSPTSIAVSQGERVTISCTASSSVNTSYLHWYQQKPGFPPRLLVYGTSSLPSGVPARFSGSGSGTSYTLTISSMEPEDAAHYYCQQGHTIPLTFGGGTKVEIK

SEQ ID NO: 75 - protein, 7 aa, Macaca fascicularis
SSVNTSY

SEQ ID NO: 76 - DNA, 32 bp, Pseudomonas aeruginosa
ggaattccatatgaaactgaagaacaccttag

SEQ ID NO: 77 - DNA, 31 bp, Pseudomonas aeruginosa
agaagctgaagccaagtaactcgagtaacgc

SEQ ID NO: 78 - protein, 10 aa, Artificial sequence
  c-myc tag
EQKLISEEDL

SEQ ID NO: 79 - protein, 6 aa, Artificial sequence
  Polyhistidine tag
HHHHHH